(19) United States
(12) Patent Application Publication
(10) Pub. No.: US 2020/0041449 A1
(43) Pub. Date: Feb. 6, 2020

(54) APPARATUSES, METHODS AND SYSTEMS FOR AUTOMATED PROCESSING OF NUCLEIC ACIDS AND ELECTROPHORETIC SAMPLE PREPARATION

(71) Applicant: Sage Science, Inc., Beverly, MA (US)

(72) Inventors: Ezra Solomon ABRAMS, Newton, MA (US); Todd J. BARBERA, Marblehead, MA (US)

(21) Appl. No.: 16/339,648

(22) PCT Filed: Oct. 4, 2017
(86) PCT No.: PCT/US2017/055193

§ 371 Date: Apr. 4, 2019

Related U.S. Application Data
(60) Provisional application No. 62/404,112, filed on Oct. 4, 2016.

(51) Int. Cl. G01N 27/447; B01J 19/00; C12N 15/10

(52) U.S. Cl. CPC G01N 27/447; C12N 15/1017; B01J 19/0046

(57) ABSTRACT

Various implementations disclosed herein relate to automated processing of nucleic acids and electrophoretic sample preparation. An exemplary disposable cassette for automated molecular processing may include a base housing, a central channel arranged in the housing, and an elution module configured to be received in central channel and to divide the central channel into a first chamber and a second chamber. The elution module comprises a housing having a proximal side, a distal side and an elution module channel passing from the proximal side to the distal side. The elution module also comprises a first membrane attached to a proximal side of the elution module, a second membrane attached to a distal side of the elution module, and a porthole in fluid communication with the elution module channel and configured for receiving a sample.

### CROSS-REFERENCE TO RELATED APPLICATIONS

This application is a non-provisional of and claims priority to U.S. Provisional Application No. 62/404,112, filed Oct. 4, 2016, and entitled “Apparatuses, Methods and Systems for Automated Processing of Nucleic Acids and Electrophoretic Sample Preparation.” All of the aforementioned applications are herein expressly incorporated by reference in their entireties.

### FIELD OF THE INVENTION

Some embodiments of the present disclosure present apparatuses, methods and systems for automated processing of nucleic acids, as well as electrophoretic sample preparation.

### SUMMARY OF SOME OF THE EMBODIMENTS

In some embodiments, systems, methods and devices are provided which include reagents, a disposable cassette, an instrument, and protocols for purification of DNA starting with intact cells. In some embodiments, a disposable cassette is provided which includes a base, a central channel, and an elution module. The elution channel is configured to divide the central channel into a first chamber and a second chamber. The elution module may comprise a first and second membrane. The first membrane may be attached to a proximal side of the elution module and traverse the central channel, thereby forming an end of the first chamber. The second membrane may be attached to a distal side of the elution module and traverse the central channel, thereby forming an end of the second chamber. The elution module may be configured to receive a sample between the proximal side and the distal side.

In some embodiments, a disposable cassette for an automated molecular process apparatus includes a base housing, a central channel arranged in the housing, and an elution module configured to be received in the central channel and to divide the central channel into a first chamber and a second chamber. The elution module may comprise an elution module housing having a proximal side, a distal side, and an elution module channel passing from the proximal side to the distal side. A first membrane may be attached to a proximal side of the elution module. The proximal side of the elution channel traverses the central channel and forms an end of the first chamber. A second membrane may be attached to a distal side of the elution module, with the distal side of the elution module being parallel to the proximal side of the channel and forming an end of the second chamber. The elution module also includes a porthole that is in fluid communication with the elution module channel and is configured for receiving a sample.

The base may have slots, and the cassette may further comprise at least two electrode holders that are configured to fit within the slots. The electrode holders may be configured to receive electrodes such that at least one electrode is arranged within the first chamber and at least one electrode is arranged within the second chamber. In some embodiments, the first and second membranes are configured to pass molecules upon application of current thereto.

In some embodiments, the first membrane is more porous than the second membrane. The second membrane may be configured to retain nucleic acid molecules. The nucleic acid molecules may comprise DNA.

The elution module may be comprised of plastic. The first and second membrane may be heat bonded to the plastic of the proximal and distal sides of the elution module, respectively. The first and second membranes may be configured to substantially block fluid flow.

In some implementations, the first chamber and the second chamber contain a buffer solution.

The elution module may further comprise openings configured to receive fasteners to affix the module to the cassette. The elution module may be configured for clamping attachment to the cassette.

An elution module may be provided for a disposable cassette used in an automated molecular processing apparatus, wherein the cassette includes a central channel arranged therein for which the elution module is placed to divide the channel into a first chamber and a second chamber. The module includes a housing having a proximal side, a distal side, and an elution module channel passing from the proximal side to the distal side. The module also comprises a first membrane attached to a proximal side of the elution module, where the proximal side of the elution module forms an end of the first chamber, and a second membrane attached to a distal side of the elution module, where the distal side of the elution module is parallel to the proximal side of the channel and forms an end of the second chamber. The elution module also includes a porthole that is in fluid communication with the elution module channel and is configured for receiving a sample.

The porosity of the first membrane may be greater than the porosity of the second membrane. The second membrane may be configured to retain nucleic acid molecules. The nucleic acid molecules may comprise DNA.

The housing may be comprised of plastic and the first membrane and the second membrane may be heat bonded to respective sides of the housing. The first and second membranes may be configured to substantially block fluid flow. The first and second membranes may be configured to pass molecules upon the application of current thereto.

The elution module may have openings that are configured to receive fasteners to affix the module to the cassette. The housing may be configured for clamping attachment to the cassette.

A method for preparing a cassette may include providing a base having a central channel having a first end and a second end. An elution module is also provided, wherein the elution module has a central plastic piece, a first membrane attached to a first side of the central plastic piece, and a second membrane attached to a second side of the central plastic piece. At least two electrode holders may be provided, each having a wire connected thereto. A casting dam that is configured to block a portion between the first end of the central channel and the first membrane is also provided, as is a cover that is configured to cover at least a portion of the central channel. The elution module may be attached to the base, the elution module spaced apart from the first end and the second end. The first end faces the first end of the central channel, and the second membrane faces the second end of the central channel. The casting dam may be placed to abut the first end of the central channel to create gap between a distal end of the casting dam and the first membrane. The gap may be casted by filling the gap with agarose and allowing the agarose to gel. The casting dam may be removed to reveal a portion of the central channel between the first end and the agarose gel. The first electrode holder may be attached between the first end of the central channel and the first membrane, and the second electrode holder may be attached between the second end of the central channel and the second membrane. The portion of the channel and an area between the second membrane and the second end of the central channel may be filled with electrophoresis buffer, and the cover may be attached to the base.

A sample may be inserted into the elution module, wherein the sample includes target molecules. A current may be applied via the electrode holders, which causes at least the target molecules to move towards the first membrane. The target molecules may be collected at or near the first membrane.

### BRIEF DESCRIPTION OF THE DRAWINGS

FIGS. 1A-C show various cut-away views of a device according to some embodiments.

FIGS. 2A-F show a device overview according to some embodiments.

FIGS. 3A-B show an elution module according to some embodiments.

FIGS. 4A-B show a base and elution module according to some embodiments.

FIGS. 5A-C show an electrode holder, base, and elution module according to some embodiments.

FIGS. 6A-B show a device according to some embodiments.

FIGS. 7A-C show a device with a lid according to some embodiments.

FIGS. 8A-D show a device and elution module according to some embodiments.

FIG. 9A shows a device according to some embodiments.

FIG. 9B shows a device with a pipette used to add agarose according to some embodiments

FIG. 9C shows a device with added agarose according to some embodiments.

FIG. 10A shows a device with a first buffer added, according to some embodiments.

FIG. 10B shows a device with a second buffer added, according to some embodiments.

FIG. 10C shows a device with a first and second buffer and electrodes in the buffer chambers, according to some embodiments.

FIGS. 11A-C show a device according to some embodiments.

FIG. 12 shows a device capable of running four samples simultaneously, according to some embodiments.

FIG. 13 shows a device capable of running four samples simultaneously, according to some embodiments.

FIG. 14 shows a table for size fractionation of purified DNA using a one dimensional device, according to some embodiments.

FIG. 15 shows an example lane sample for size fractionation of purified DNA using a one dimensional device, according to some embodiments.

FIG. 16 shows a table for size fractionation of purified DNA, according to some embodiments.

FIG. 17 shows an example size fractionation of purified DNA, according to some embodiments.

FIG. 18 shows data for an example size fractionation of purified DNA, according to some embodiments.

FIG. 19 shows a table for isolation of bacterial DNA, according to some embodiments.

FIG. 20 shows a table for isolation of bacterial DNA, according to some embodiments.

FIG. 21 shows a table for isolation of bacterial DNA, according to some embodiments.

FIGS. 22A-B show examples of isolation of bacterial DNA, according to some embodiments.

FIG. 23 shows an example of an isolation of bacterial DNA, according to some embodiments.

FIG. 24 shows an example isolation of high mol wt DNA from white blood cells, according to some embodiments.

FIG. 25 shows an example isolation of high mol wt DNA from white blood cells, according to some embodiments.

FIGS. 26A-B show an example isolation of high molecular weight DNA from white blood cells, according to some embodiments.

FIG. 27 shows an example isolation of high molecular weight DNA from white blood cells, according to some embodiments.

FIG. 28 shows a cutaway view of a device, according to some embodiments.

FIG. 29 shows another view of the example size fractionation of purified DNA of FIG. 17, according to some embodiments.

FIGS. 30A-E show top-view schematics of a HMW DNA extraction workflow, according to some embodiments.

FIGS. 31A-K show top-view schematics of a workflow, according to some embodiments.

FIGS. 32A-D show top-view schematics of a size selection workflow, according to some embodiments.

### DETAILED DESCRIPTION OF SOME OF THE EMBODIMENTS

Apparatuses, systems, and methods described herein include reagents, a disposable cassette, an instrument, and protocols for purification of DNA starting with intact cells. These apparatuses, systems, and methods demonstrate purification of high molecular weight genomic DNA from either mammalian white blood cells or lysozyme treated E coli cells, as well as size fractionation of DNA starting with purified DNA.

In some embodiments, the apparatuses, systems, and methods described herein include a simple, low cost disposable (“cassette”), ability to handle large or small amounts of sample, suitability for use as either a manual system with one or a few samples, or as an automated system suitable for large numbers of samples.

Multiple, sequential enzymatic reactions may be performed. During such reactions, the DNA remains embedded in an agarose matrix. This may allow for the ability to use either liquid handling (pipetting) or electrophoresis to add and remove reagents such as enzymes, cofactors or buffers. Further, since the DNA may remain embedded in an agarose matrix, intermediate purification steps using particles, such as SPRI beads, or other processes such as Ethanol precipitation, may not be needed, thus avoiding the complexity, cost and loss of sample with such protocols.

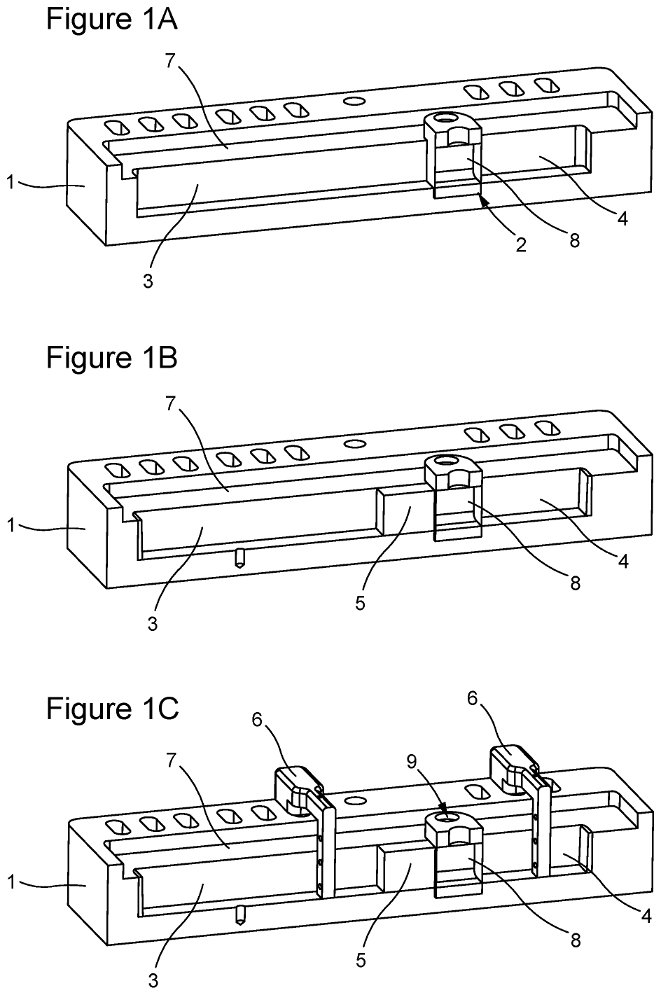

FIG. 1A shows a base 1 fitted with an elution module 2. The elution module 2 divides the central channel into two compartments 3, 4. The two membranes bound a sample compartment 8. In FIG. 1B, a block of agarose 5 is cast next to the elution module 2 and buffer is added to fill the chambers 3, 4. If the buffer level is below the shelf 7, there is no bulk flow between the chambers 3, 4, but if the buffer is higher than shelf 7, liquid can flow between the buffer chambers 3, 4. Nonetheless, there is a continuous fluid path for electrophoresis, as the membranes are permeable to ions and current. FIG. 1C shows electrode holders 6 with platinum wire added to the configuration(s) shown in FIGS. 1A-B. The platinum wire is connected to a power supply. A sample is added to the sample compartment 8 via porthole 9.

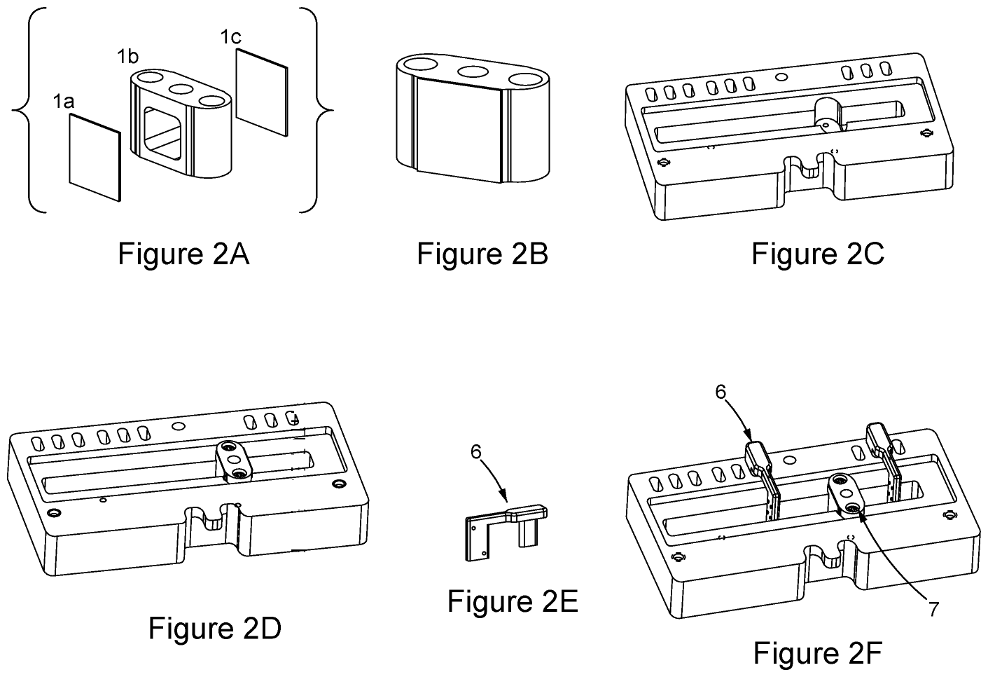

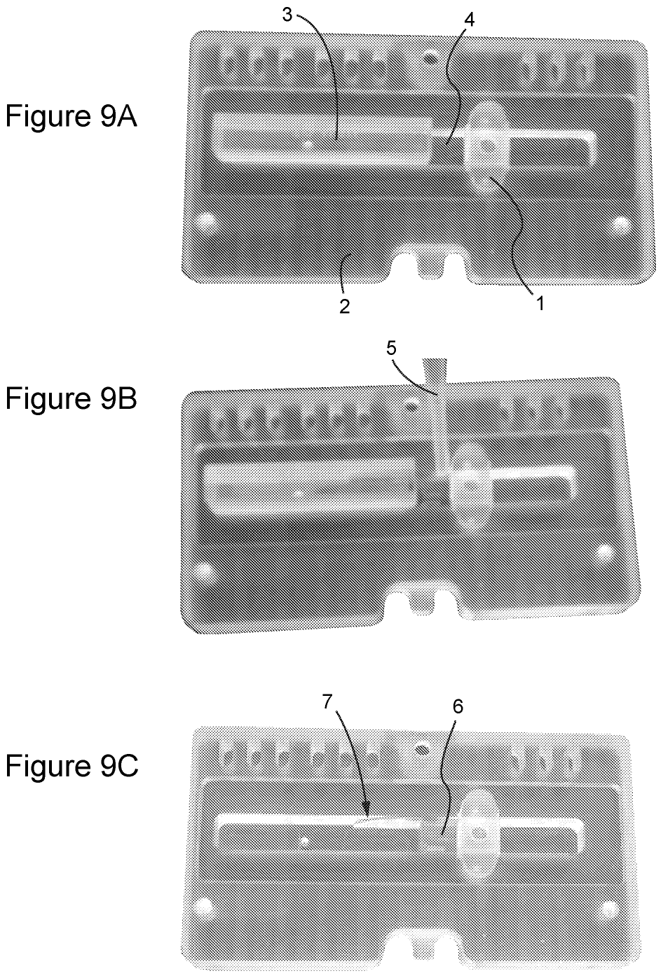

As shown in FIGS. 2A-F, the cassette may consist of an elution module, a base, and electrode holders. FIG. 2A shows an exploded view of the elution module showing membranes 1A, 1C and acrylic elution module 1B. The assembled elution module is shown in FIG. 2B, where the membranes (1A, 1C) are sealed the acrylic elution module (1C) by heat bonding. An exemplary base is shown in FIG. 2C, while FIG. 2D shows the base with the elution module (of FIG. 2B) inserted therein. In some embodiments, the elution module may be held down by two screws, as shown in FIG. 2D. FIG. 2E shows an electrode holder 6. The electrode holder of FIG. 2E may be inserted into the base, as shown in FIG. 2F. As shown, several electrode holders 6 may be inserted into the base. In an exemplary embodiment, one or more electrode holders may be placed on a first side of the elution module, and one or more electrode holders may be placed on a second side of the elution module. Casting dams may also be provided (FIG. 9) to allow casting of agarose gels in the cassette.

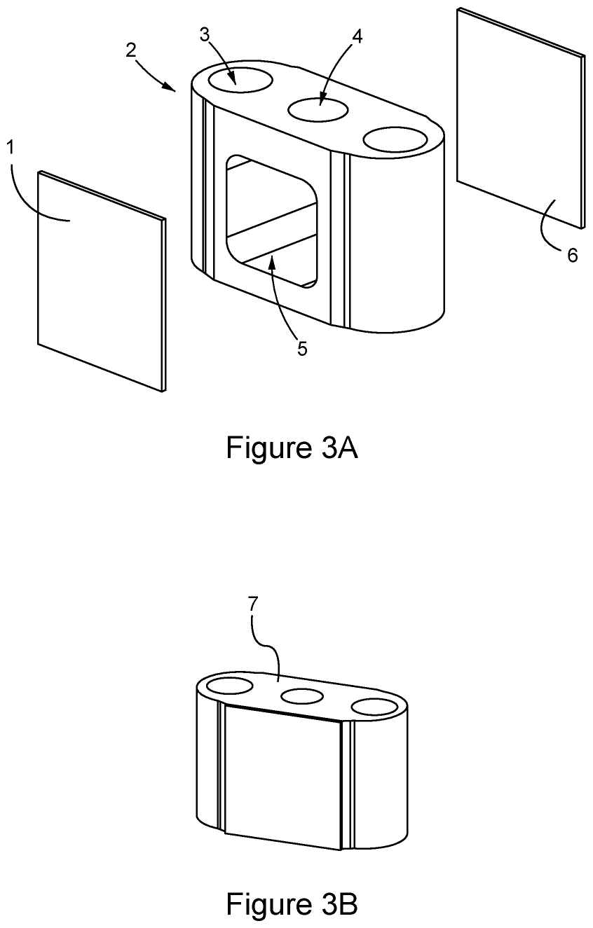

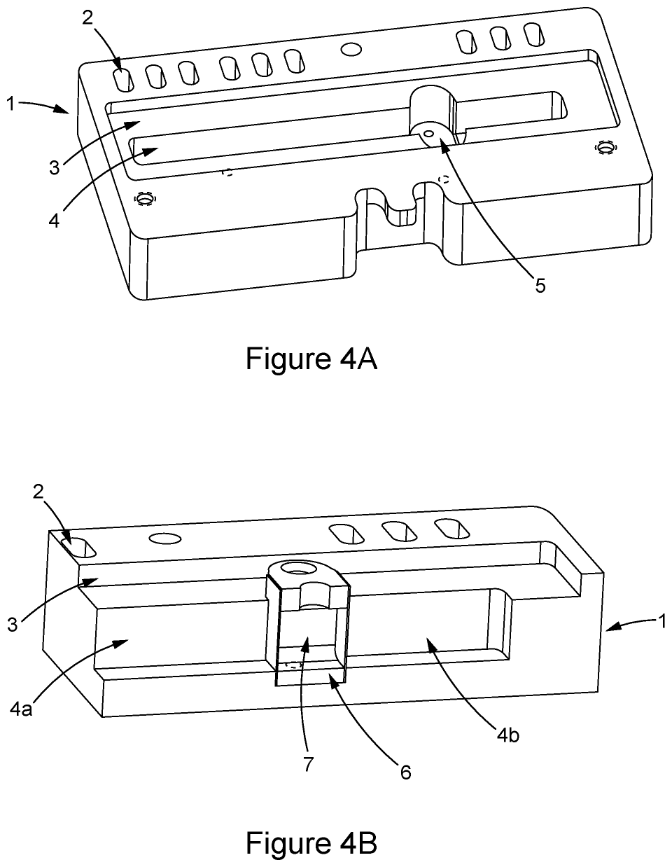

The elution module, as shown in FIG. 3, may consist of two rectangular pieces of membrane which are heat bonded (and/or otherwise attached) to a central plastic piece. The first membrane 1 may allow passage of DNA and protein, such as Durapor. The central piece may comprise the elution module body 2. The elution module body may be machined acrylic. The body 2 may have at least one hole 3 configured to pass a screw, such as an M2 or M3 screw. The screws may be used to hold the elution module in place when the elution module is screwed into the body (e.g., FIG. 2C). The body may also have a porthole 4 and a central channel 5. A second membrane 6 may be configured to retain DNA molecules, such as, for example, a membrane having a 10 kd cutoff PES. When assembled, as shown in FIG. 3B, the membranes 1, 6 enclose the channel 5 to form a space that is bounded on two sides by membrane. This is shown in cross section in FIG. 4B (6).

As discussed above, the two membranes, which are bonded to the elution module, may have different properties. One membrane is chosen to retain molecules of interest. For example, a first membrane may be a PES (poly ether sulfone) membrane rated to retain molecules larger than 10,000 Daltons in mass, and the other membrane may be chosen so that DNA molecules can pass through the membrane, e.g., a membrane rate to have pores with a nominal 0.5 micron size.

The nominal or rated properties of the membranes may vary. Both membranes may be permeable to water and ions, so that the electric field can pass through the elution module. One membrane may retain molecules of interest, while the other membrane may be relatively porous, as explained below.

In some embodiments, a porous membrane, may be Durapor PVDF HVPP membrane (EMD Millipore Corporation, Chicago Ill. 60673) with 0.45 micron pores, or Durapor with 5.0 micron pores (Millipore type SVPP, Catalog Number: SVLP09050). The nonporous membrane may be Biomax PES, catalog SF1J007A1. Other membranes that may be used, including membranes of regenerated cellulose. Some such membranes are described in Millipore: Ultrafiltration Membranes: Ultrafiltration membranes for Macromolecule Processing Product Selection Guide, April 2008, Millipore Corporation, which is incorporated herein by reference in its entirety.

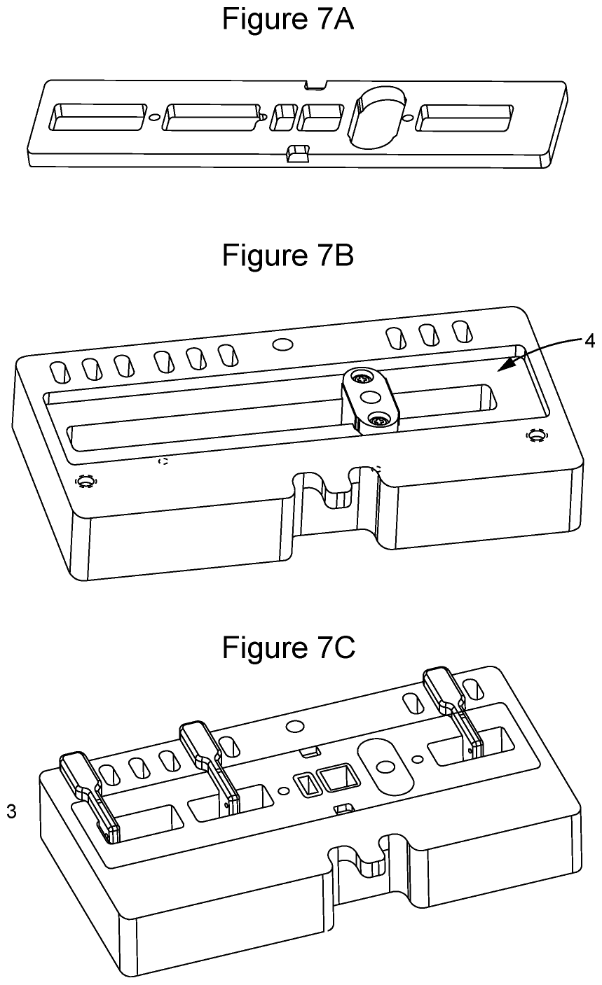

As shown in FIG. 3A-B, the elution module has a porthole 4 which allows liquid to be added or removed from the membrane bounded space; the module also has holes 3 which allow the module to be attached to the base with screws (as shown in FIGS. 2D, 2F, 7).

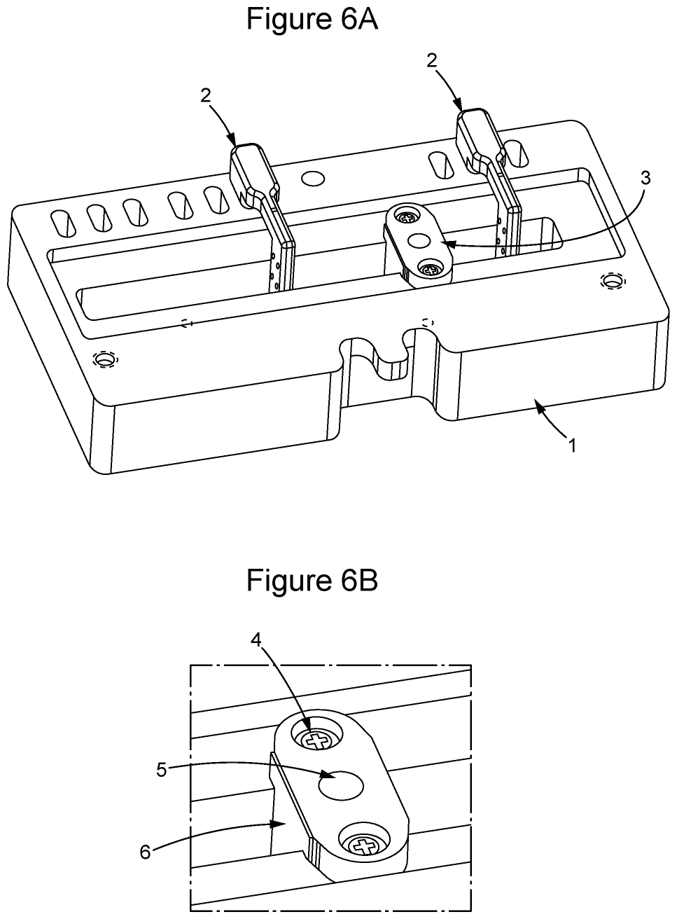

Electrode holders are fitted with platinum wire, and are inserted into slots in the base (FIGS. 2F, 6).

As shown in FIG. 4A, the base 1 may have a central channel 4, an oval slot 5 for holding the elution module, and slots 2 to hold the electrode holders. The base may also have a cutout 3 for a lid.

FIG. 4B shows an elution module 6 inserted in the base 1. The central channel in the base is divided into two buffer chambers 4a and 4b by the elution module 6. In the elution module 6, there is a sample compartment 7 bounded by membranes. When liquid is added, the buffer chambers 4a and 4b, and the sample compartment 7 form a linear liquid path. The membranes substantially block bulk fluid flow, but allow ions and other molecules to pass when current is applied.

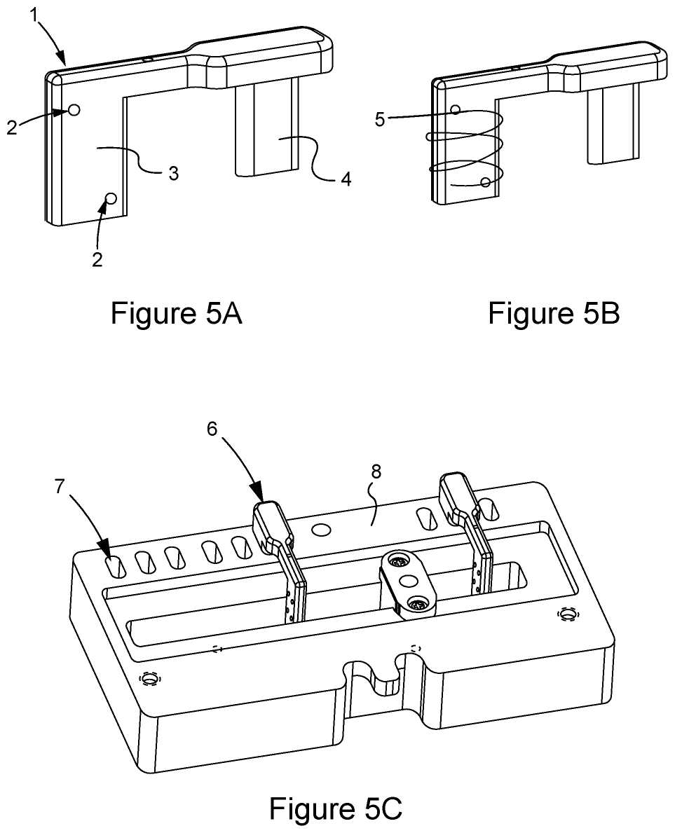

FIG. 5A shows an electrode holder 1 according to some embodiments. The electrode holder 1 may be configured with a tab 3 for holding electrode wire, which may also have at least one hole 2 to receive the electrode wire. FIG. 5B shows the tab with wire 5 wrapped around the tab and through holes 2. In some embodiments, the electrode wire is platinum. The electrode holder 1 may also be configured with a tab 4 (FIG. 5A, B) that is configured to fit in one of the slots 7 the base 8, as shown in FIG. 5C.

FIG. 6A shows the base 1 with electrode holders 2 and elution module 3 inserted therein. FIG. 6B shows a membrane 6 affixed to the elution module 3, and the elution module 3 is held in place using screws 4. As shown, the elution module 3 has a porthole 5.

FIG. 7A shows a lid 1 (also referred to herein as a “cover”) that is configured to fit into the base 2 shown in FIG. 7B. In some embodiments, the lid may cover substantially all of the base. In the shown embodiment, the cover 1 is configured to fit in an inset portion of the base 1. The base 100 may have an opening configured to fit around the elution module and openings configured to receive the electrode holder tabs 3 (FIG. 5A). FIG. 7C shows the base configured with the elution module, the cover, and the electrode holders.

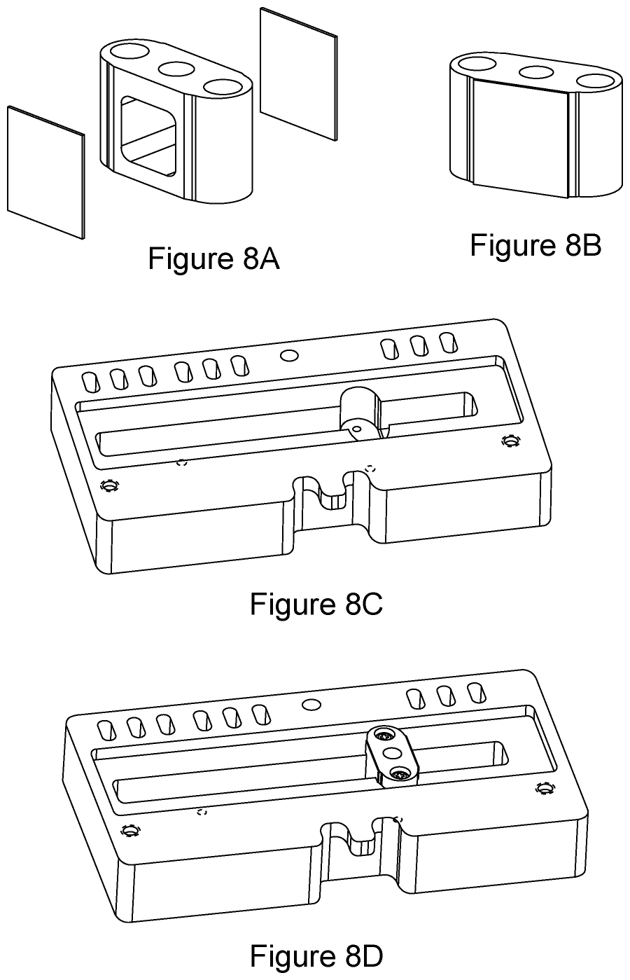

FIGS. 8A-D show an embodiment of an exploded view of an elution module (FIG. 8A), the elution module (FIG. 8B), a base (FIG. 8C), and the elution module configured within the base (FIG. 8D).

FIG. 9A shows a device ready for casting of agarose. The elution module 1 is inserted into the base 2, and fitted with a casting dam 3. In some embodiments, the casting dam 3 may be sized to make a gap 4 of about 1 cm between the casting dam 3 and the elution module 1. As shown in FIG. 9B, a Pasteur pipette 5 may be used to add agarose to the space 4 between the casting dam 3 and elution module 1. FIG. 9C shows that the casting dam 3 has been removed, and a block of agarose 6 remains in the gap 4. Any extra agarose 7 may be trimmed before use.

In some embodiments, an elution membrane may be inserted into the base (FIG. 1A) and a block of agarose may be cast (FIG. 9B and Example 1) by adding a casting dam 3 (FIG. 9) and then molten agarose solution; the agarose gels to from a hydrogel that is continuous with the adjacent membrane. As noted above, the two membranes may be different—e.g., one is relatively porous (nominal 0.5 micron pores) and one retains DNA molecules (10,000 Dalton nominal rating). The block of agarose may be cast next to the porous membrane.

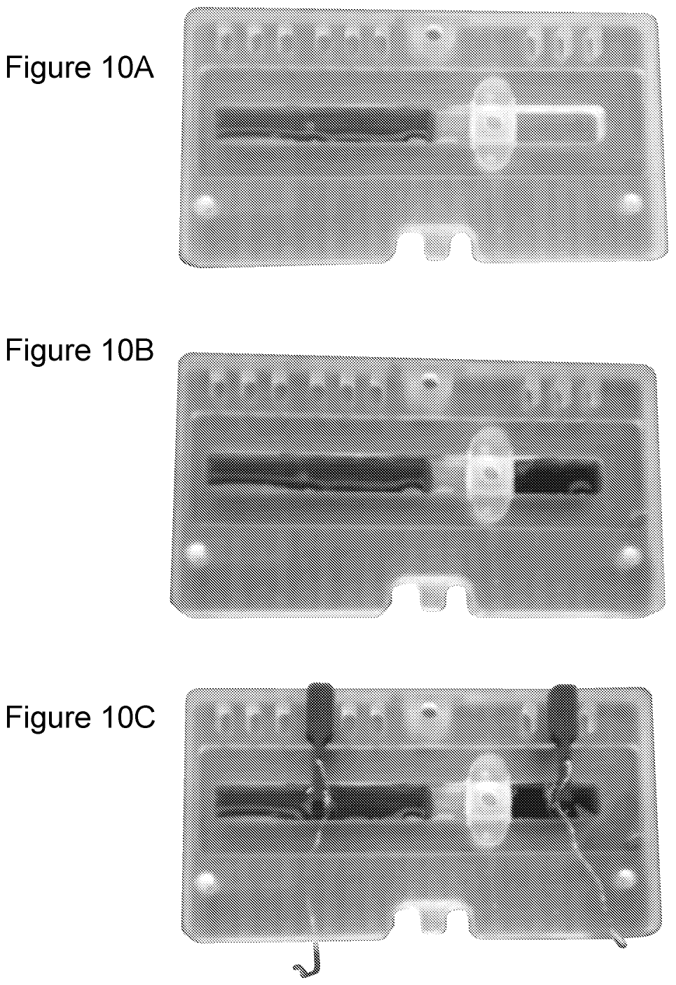

The buffer chambers 3, 4 (FIG. 1C) may be filled with electrophoresis buffer, and sample may be added through the porthole 9 into the elution module. FIG. 10A shows the buffer added after the agarose as gelled. The buffer may be added to the buffer chamber that is adjacent to the agarose. Buffer may also be added to the other buffer chamber, as shown in FIG. 10B. As shown in FIG. 10C, electrode holders (with platinum wire as conductor) may be added, and the wires are attached to a power supply.

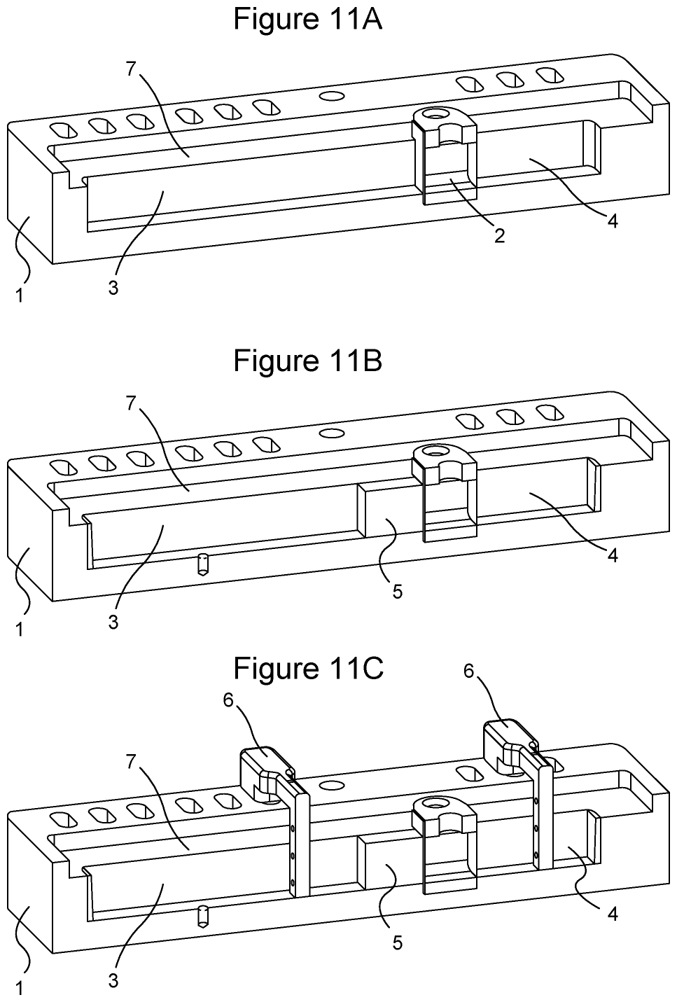

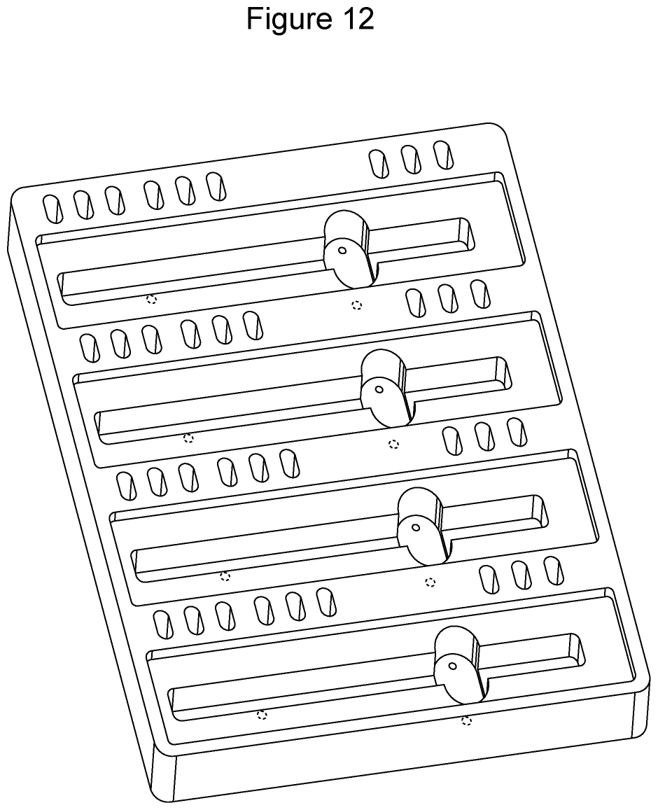

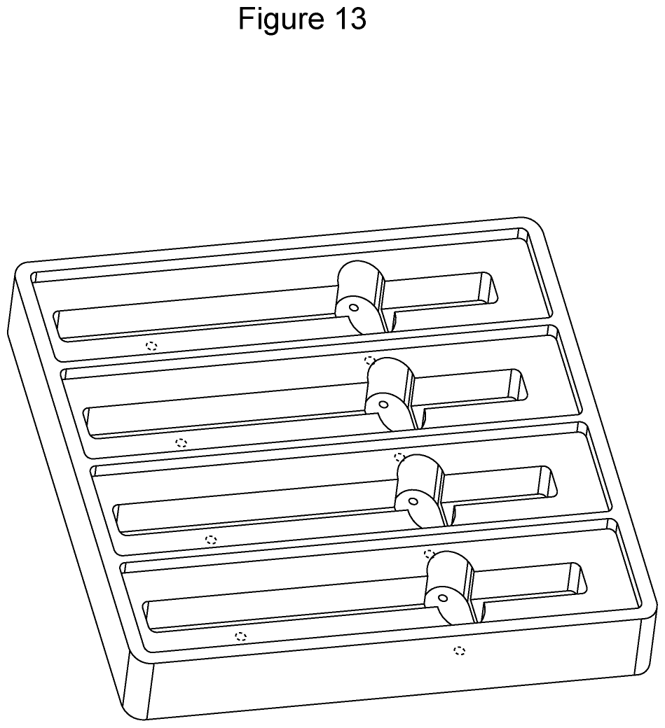

This is also shown in FIGS. 11A-C. In FIG. 11A, the base 1 is fitted with elution module 2. The elution module divides the central channel in two compartments 3, 4 such that each membrane of the elution module 2 forms an end of each compartment 3, 4. A block of agarose 5 is cast next to the elution module, as shown in FIG. 11B. As shown in FIG. 11C, the electrode holders 6 may be added. In some embodiment, one electrode holder is configured on each side of the elution module 3 and a tab of each electrode holder is inserted into its respective chamber 3, 4. In other embodiments, more than one electrode holder is configured on one or both sides. The liquid can flow between buffer chambers 3, 4 only if the liquid level exceeds the height of the shelf 7.

When current is applied, e.g., with a positive electrode in chamber 3; thus, negatively charged particles such as cells or DNA migrate toward the agarose/membrane side of the sample compartment. Since the pores in agarose gels are smaller than cells, the cells will become entangled near or at the surface of the agarose coated membrane. Sodium dodecyl sulfate (“SDS”) is then added into the elution module via the porthole (FIGS. 1C, 9) and electrophoresis continues.

As the SDS migrates toward the positive electrode, the SDS will encounter the cells entangled at the agarose membrane surface. The SDS will cause the cells to lyse and protein to become coated with SDS, and the cellular debris and SDS coated protein will migrate through the agarose gel and into the buffer chamber 3.

However, intact chromosomes, either mammalian or bacterial, may not migrate appreciably into the gel. As explained in Chapter 7, “preparation, manipulation, and pulse strategy for one-dimensional Pulsed-field gel Electrophoresis (OFPFGE)”, in “Pulsed-Field Gel Electrophoresis”, eds M Burmeister and L Ulanovsky, Huuman Press, Totowa N.J., 1992, which incorporated herein by reference in its entirety, intact mammalian or bacterial chromosomes do not migrate appreciably into agarose gels with DC fields; DNA molecules of up to 6 megabase pairs will migrate, albeit only under special conditions.

Thus, intact chromosomes will remain entangled at the agarose/membrane surface. Since the chromosomal DNA is near or at the agarose/membrane surface, it is accessible to enzymes added to the sample compartment. Such enzyme can diffuse a short distance into the agarose layer and act on the entangled DNA molecules. Diffusion of proteins in agarose gels is discussed in Pluen, Alain, et al., “Diffusion of Macromolecules in Agarose Gels: Comparison of Linear and Globular Configurations,” Biophysical Journal, Volume 77, July 1999, pp. 542-552, incorporated herein by reference in its entirety (see FIG. 2, which shows that proteins of up to 100,000 Daltons will diffuse into agarose gels). Thus, addition of an enzyme that makes double strand breaks in DNA will convert the immobile, entangled chromosomes into mobile, entangled shorter fragments; i.e., as long as the fragments are less then approximately 2 megabase pairs, they will show some mobility during agarose gel electrophoresis.

Thus, after adding cells and then SDS, and then enzyme, cut DNA can be recovered in the elution module by electrophoresis with the positive electrode in the buffer chamber 4 (FIG. 1A). The DNA molecules will migrate out of the gel and into the sample compartment and migrate toward the non-agarose coated membrane. As noted above, this membrane is chosen so that DNA molecules are retained in the sample compartment, and do not pass through the membrane.

### Elution Module Example

As shown in FIGS. 3A-B, the elution module may have a plastic body (2), with a central channel 5, holes 3, and a porthole 4. The membranes 1 and 6 may be heat staked to the plastic body to produce an assembled elution module 7. A cross section of an elution module inserted in a base is shown in FIG. 4B; the sample compartment 7 may be bounded on both sides by membrane and accessible via the porthole.

The elution module may be affixed to the base, which can be done by, e.g., gluing, ultrasonic welding, press fit, etc. For the specific examples below, the elution module has been fixed to the base in two different ways.

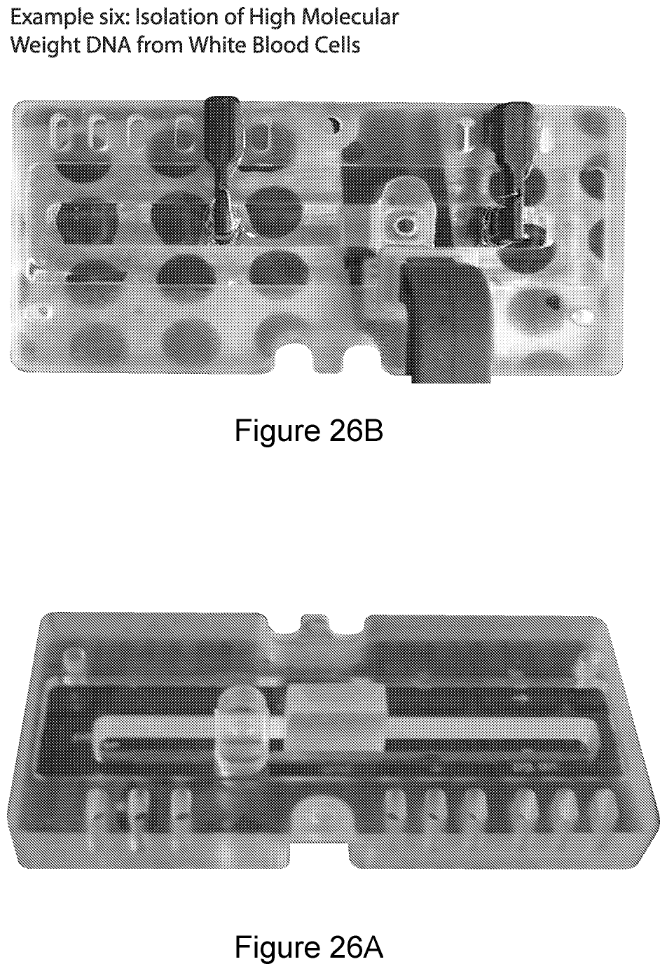

In the first method, screws were used. As shown in FIGS. 6A-B, holes (FIG. 3, 3) allow two nonconductive screws 4 (nylon M2) to pass through the elution module into threaded holes in the base. Alternatively, an elution module without holes can be fixed to the base with a clamp. This is shown in FIG. 26.

As noted above, the membranes 1,6 (FIG. 3) may be different. One membrane may be chosen so that DNA and protein molecules can transit through the membrane relatively unhindered. In some embodiments, Durapor PVDF HVPP membrane (EMD Millipore Corporation, Chicago Ill. 60673) with 0.45 micron pores may be used; in other embodiments, Durapor with 5.0 micron pores (Millipore type SVPP, Catalog Number: SVLP09050 may be used. The second membrane may be chosen to retain, but not bind, molecules of interest. In some embodiments, Biomax PES, catalog SF1J007A10 may be used. The membrane is bonded so that the size selective PES surface is on the inside, facing the sample compartment.

In some embodiments, prior to use, the PES membrane is made hydrophilic, so that air bubbles are not trapped when buffer is added. For example, drop of glycerol ethanol solution (equal parts by weight of glycerol and ethanol) may be added to the outer surface of the PES membrane, and the elution module is allowed to sit at room temperature for at least several hours.

The following Examples correspond to at least some of the embodiments disclosed above, the steps/processes also correspond to further embodiments.

### Example 1

Cassette Assembly and Casting Agarose

An elution module with Durapor and PES membrane was prepared by first treating the PES with glycerol/ethanol solution; after a few hours, the elution module was filled with buffer (0.5×KBB, sage science; 0.5×KBB contains 51 mM Tris base; 24 mM Taps; 0.08 mM EDTA), using a pipette to add liquid through the porthole into the central compartment.

To demonstrate that the membranes are firmly bonded to the elution module, slight pressure was applied by pressing on the liquid at the top of the porthole.

The buffer is aspirated using a pipettor, and the elution module carefully dried by blotting the plastic and Durapor with a paper towel; the PES surface was not touched.

It is thought that if the Durapor is dry, then when molten agarose solution is added, the agarose will be taken up by capillary action into the Durapor, thereby forming a durable, tight seal between the agarose and the membrane.

FIG. 9A shows a base 2 with an elution module 1 and a casting dam 3. The casting dam is sized so that the gap 4 between the dam and the elution module is 10 mm.

FIG. 9B shows a molten agarose solution (0.75% wt/v seakem gold agarose (Lonza), in 0.5×KBB buffer (Sage Science; the agarose is dissolved by heating and the solution stored at 65 degrees centrigrade for up to several days prior to use) being added with a disposable pasteur pipette 5.

The agarose is added to be level with the shelf 3 (FIG. 4A-B, see also FIGS. 1A-C, 7, FIGS. 11A-C, 7).

After the agarose cools to form a gel, the casting dam is removed (FIG. 9C), and extra agarose 7 which filled the thin space between the casting dam and the base is removed with a disposable scalpel.

In this example, the elution module was simply pressed into the base; an identical procedure is used for modules that have screw holes, except that the module is fixed to the base with two screws.

### Example 2: Size Fractionation of Purified DNA Using a One Dimensional Device

In this example, we demonstrate that purified DNA can be size fractionated using a simple, rapid, high throughput linear device.

### Cassette Preparation

An elution module was prepared as described in Example 1, except that it was fixed to the base with M2 screws.

Membranes are Durapore PVDF HVPP 0.45 um Roll Stock (EMD Millipore, Chicago Ill.) and PES Biomax 10 kD 27 inches SF1J007A10) Agarose was cast as described in Example 1, and after the agarose gelled, the casting dam was removed, and 0.5×KBB buffer was added to the buffer chambers (FIG. 4B, 4a 4b) and buffer was added to the sample compartment of the elution module.

Electrodes were added and connected to a Pippin Pulse power supply (Sage Science).

The device was run at 50 V DC, with the positive electrode on the Durapor side, for a few minutes to condition the device. The current (measured with a BK precision Mini-Pro Digital Multimeter Model 2405A) was 4.5 mA.

### Sample Preparation

Mix 20 microliters of lambda DNA (catalog number N3013, New England Biolabs, Ipswich Mass., 500 microgram/mL), 20 microliters of 2 log ladder (catalog number N3200, New England Biolabs, Ipswich Mass., 1,000 microgram/mL), and 410 microliters of TE buffer (TE buffer has 10 mM Tris HCl pH 7.5 and 1 mM EDTA). The DNA concentration was determined with a Qubit HS assay (Catalog number: Q32851 ThermoFisher); the result is 52 nanogram/microliter, which is 78% of the expected value based on the vendors specification.

### Sample Loading and DNA Fractionation

The elution module sample compartment was emptied using a pipette, and 430 microliters of sample, 2 microliters of Xylene Cyanol dye solution (10 milligram/mL) and 100 microliters of TE were added to the sample compartment, and the solution gently mixed.

Electrophoresis was done for forty minutes at 50 V DC, using a Pippin Pulse power supply, with the positive electrode in the buffer chamber next to the agarose coated Durapor.

At fifteen minutes, it is observed that the Xylene cyanol dye has formed a broad band in the agarose gel next to the Durapor; the band moves to the end of the gel by forty minutes. The current drops from 4.5 mA to 2.5 mA during the run.

The buffer in the elution module was recovered (Fraction 1); the elution module was rinsed with 0.5×KBB (Fraction 2); the elution module was filled with 0.5×KBB and the porthole was sealed with a rubber stopper.

The buffer chambers were rinsed twice with 0.5×KBB and then refilled with fresh buffer.

Recovery of DNA still in the agarose gel was by electrophoresis; 50 V DC for 10 min with the PES side positive, then one minute using a pulse program of 4 msec fwd/4 msec reverse (In the Pippin Pulse software, values for the waveform parameters were 4/4/0/0/0/0/1000), then 25 V DC with the PES side negative, for eight seconds, to back the DNA off the membrane.

The DNA in the elution module was recovered as fraction 3.

The Elution step was repeated 4 more times, and the material recovered as fractions 4-7.

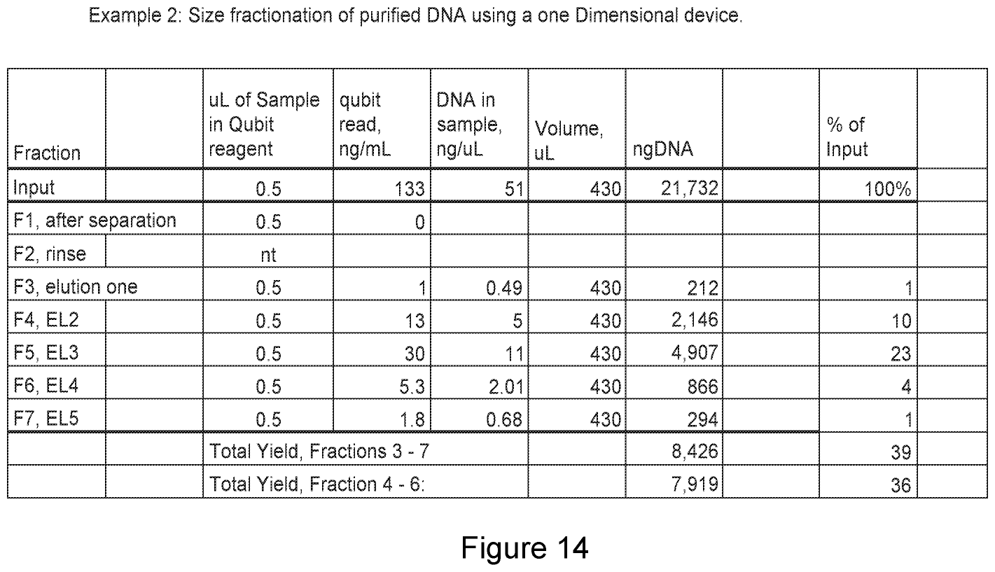

The concentration of DNA in the different fractions was determined using the Qubit HS assay—see FIG. 14.

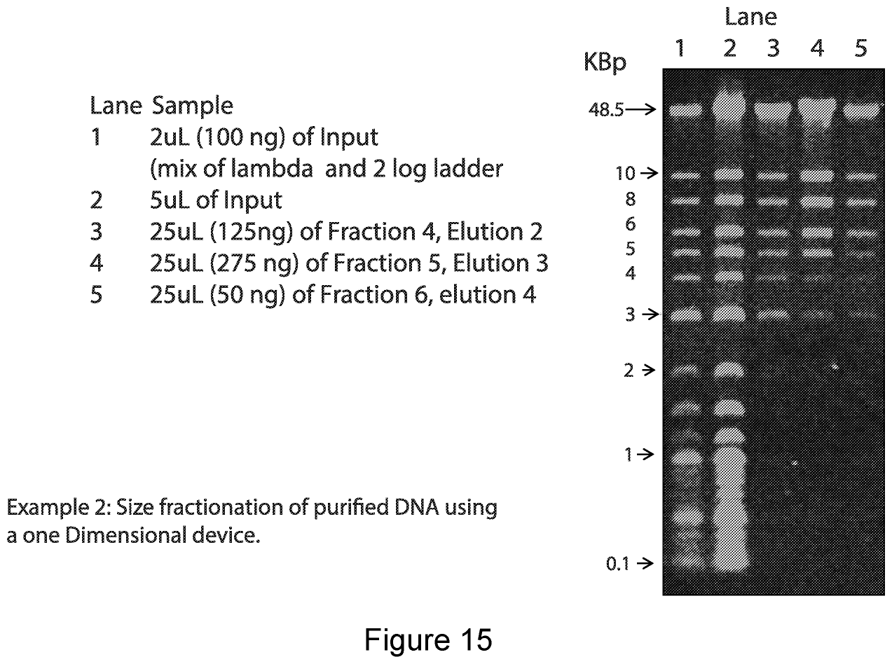

DNA was examined by agarose gel electrophoresis (0.75% seakem gold (Lonza), 0.5×KBB buffer (Sage) using a Sage Pippin Pulse power supply, 100 V DC for 120 minutes; the gel was stained with Ethidium Bromide and photographed with UV transillumination. Agarose gel of size fractionated DNA is shown in FIG. 15.

### Results

As shown by agarose gel electrophoresis, the starting material consists of fragments ranging in size from 0.1 to 48.5 KBp. If size fractionation has occurred, then there should be loss of smaller fragments. As can be seen, fragments smaller than 2 Kbp are not recovered in the eluted DNA, thus demonstrating size fractionation with a cutoff between 2 and 3 Kbp. By Qubit assay, we recovered 39% of the input sample.

### Example 3: Size Fractionation of Purified DNA

A cassette was prepared as described in Example 2.

The sample in a total volume of 450 uL, contained 7,000 nanograms of E coli genomic DNA (Lofstrand Laboratories) and 18,000 nanograms of 2 log ladder (New England Biolabs, Ipswich Mass., a series of discrete bands from 0.1 to 10 kbp in size); the DNA is diluted in TE buffer (10 millimolar Tris HCl, ph 7.5; 1 millimolar EDTA).

The sample was loaded into an elution module and 100 microliters of TE buffer was added, and after mixing 15 microliters was taken and saved as fraction 0 (input).

The DNAs were size fractionated by electrophoresis, using a pippin pulse controller, with the positive electrode on the Durapor side of the EM, using the following schedule:

Ten minutes, DC, 50 V

110 minutes, pulse field, 40 V. The pulse field was defined by the following values entered into the Pippin Pulse software: 150,50,30,10,3,1,81.

During the DC portion of electrophoresis, the volume in the elution module decreased, and at seven minutes, 240 microliters of 0.5×KBB was added.

At the end of the size fractionation step, the contents of the elution module were recovered and saved as Fraction 1, after size step.

The DNA was then eluted out of the agarose gel into the elution module.

Using the following electrophoresis schedule, with the positive electrode on the PES side

| Time min | Voltage | Waveform |
|---|---|---|
| 2 | 50 V | DC |
| 0.5 | 50 V | 4/4/0/0/0/0/1000 |
| 10 | 80 V | 300/100/30/10/30/10/45 |
| 0.5 | 50 V | 4/4/0/0/0/0/1000 |
| 8 sec | −25 | DC (the PES side is negative) |

The material in the elution module was recovered and saved as Fraction 3, 1st elution.

The elution process was repeated three more times.

### Results

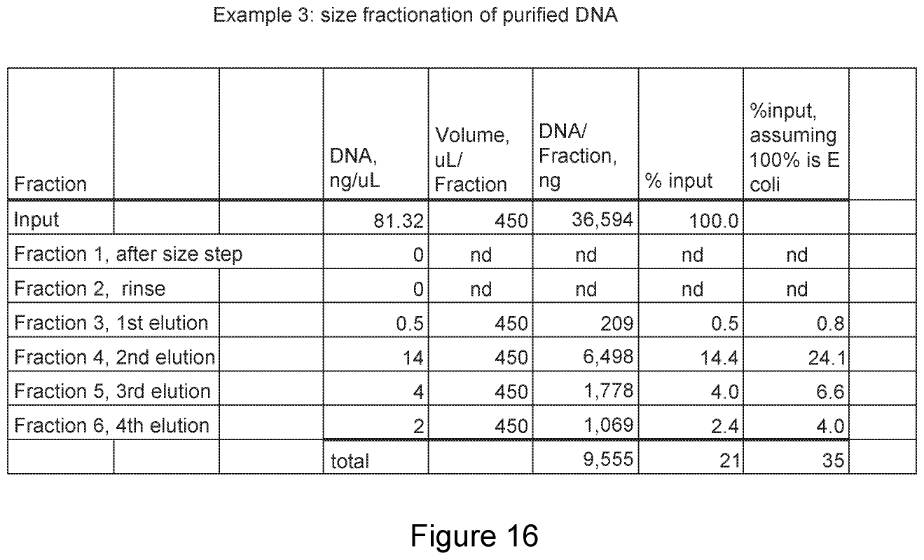

Qubit assay—FIG. 16.

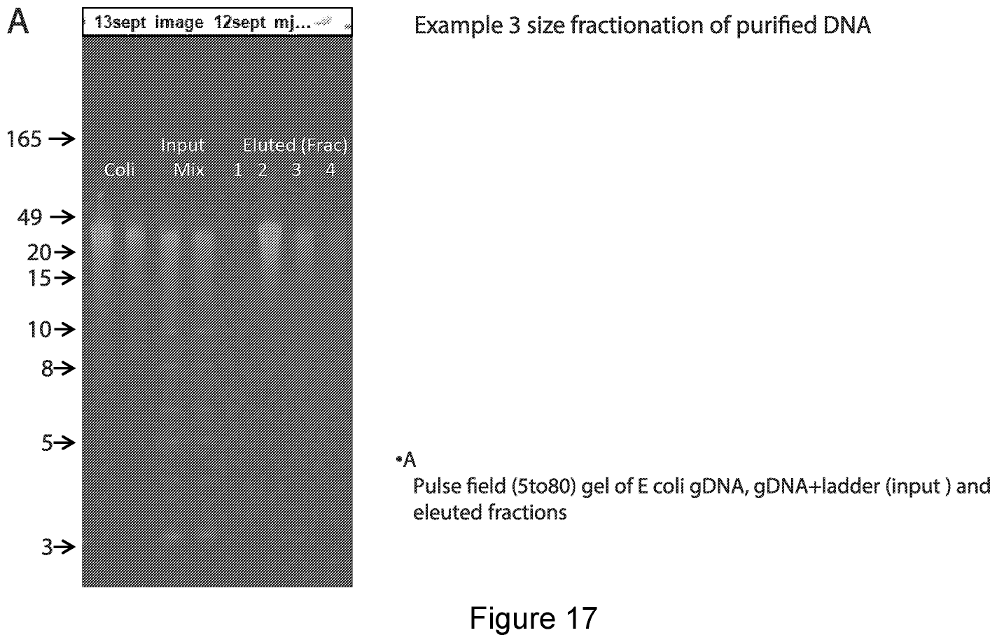

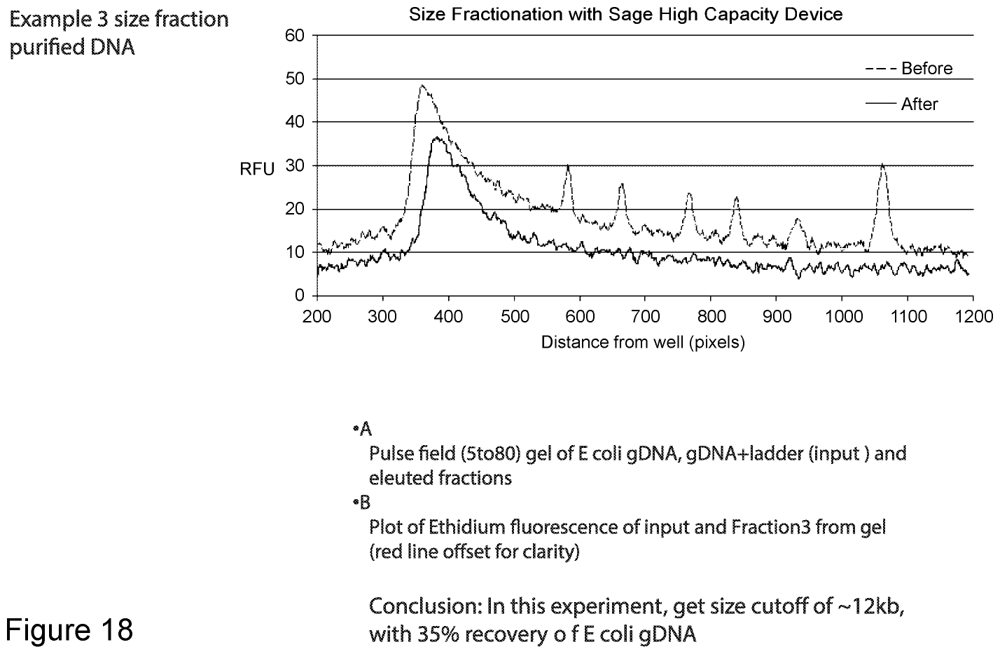

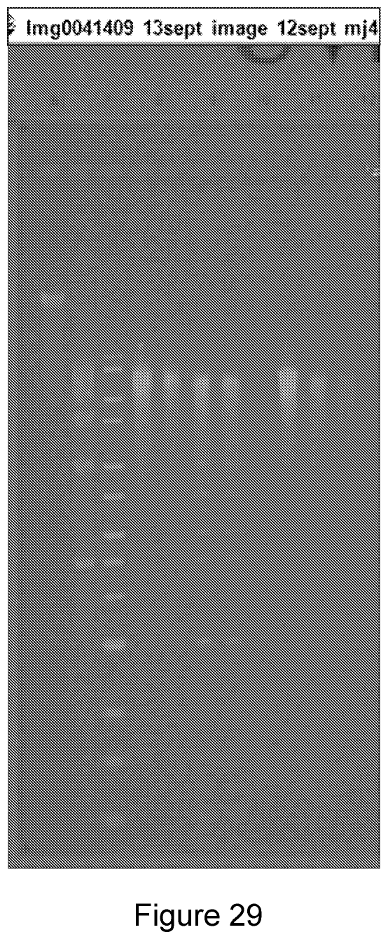

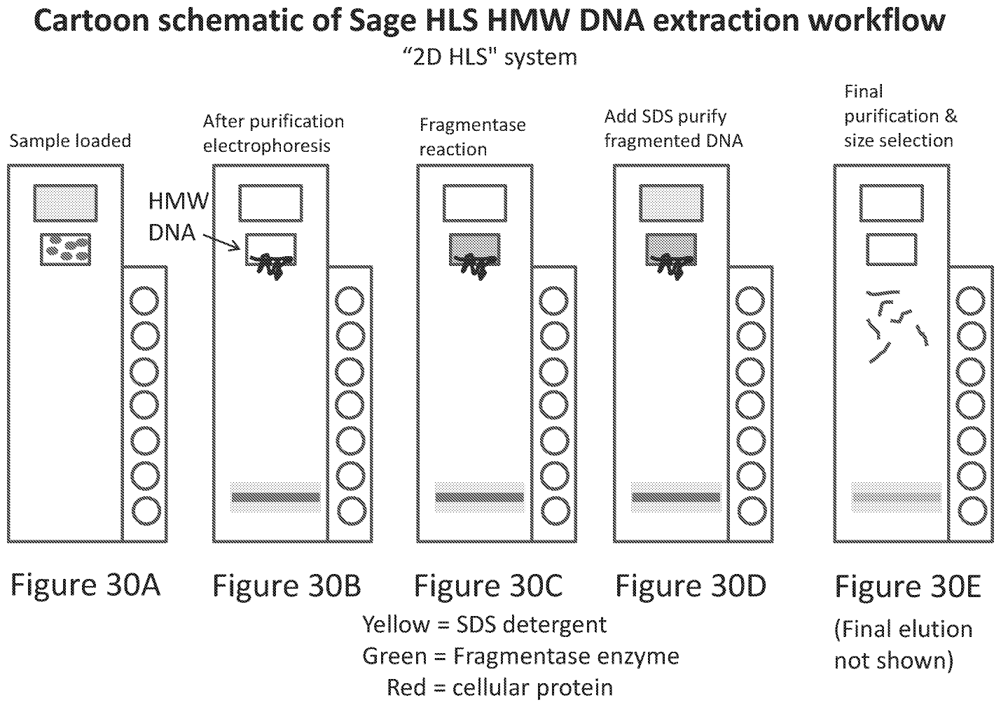

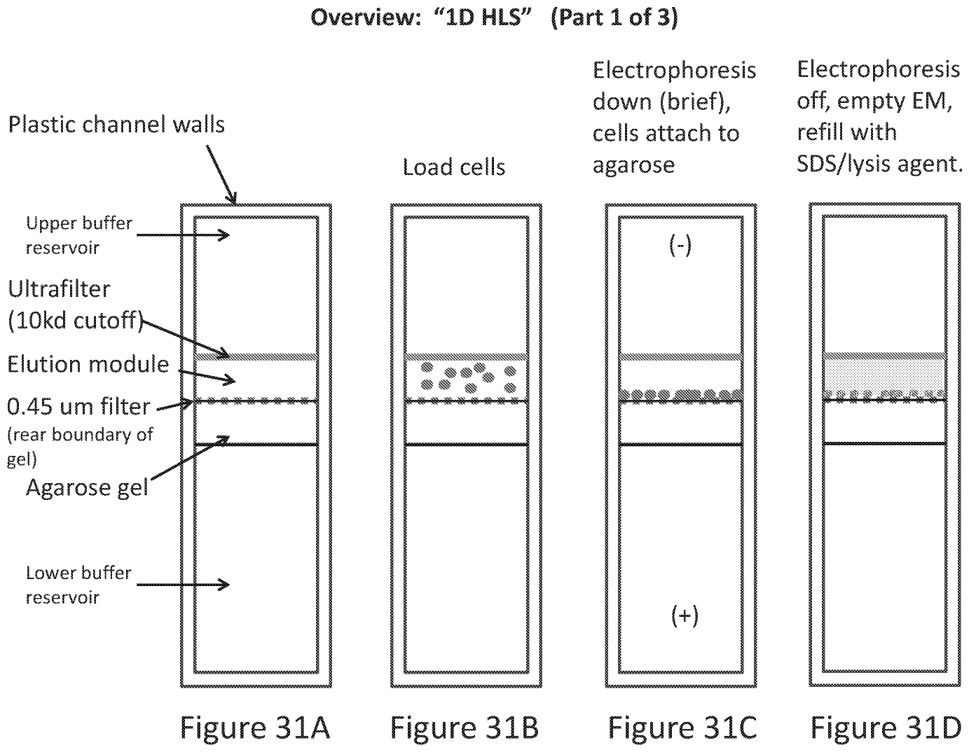

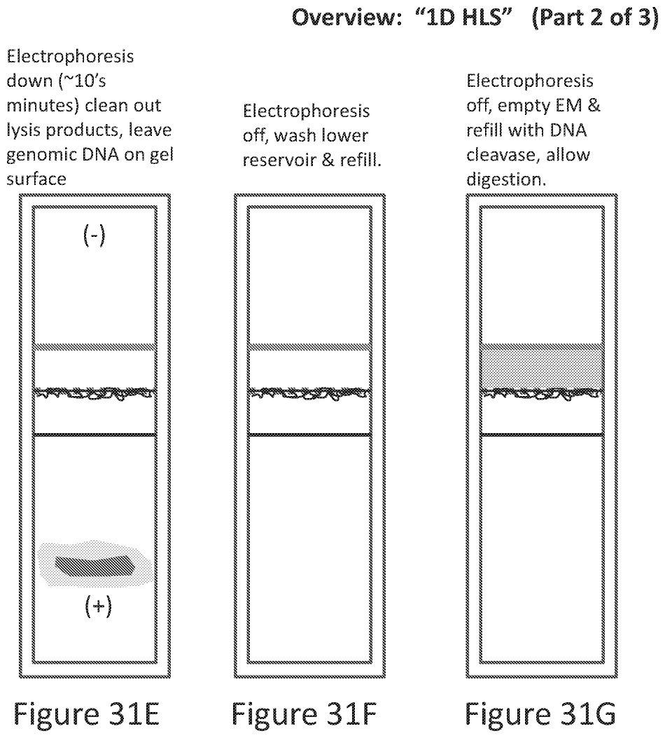

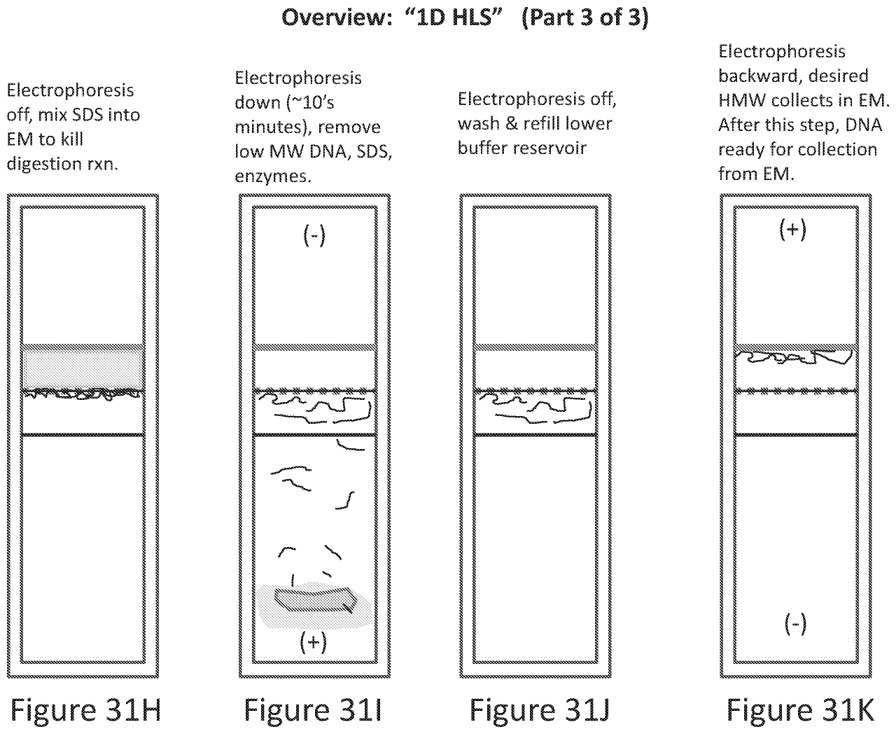

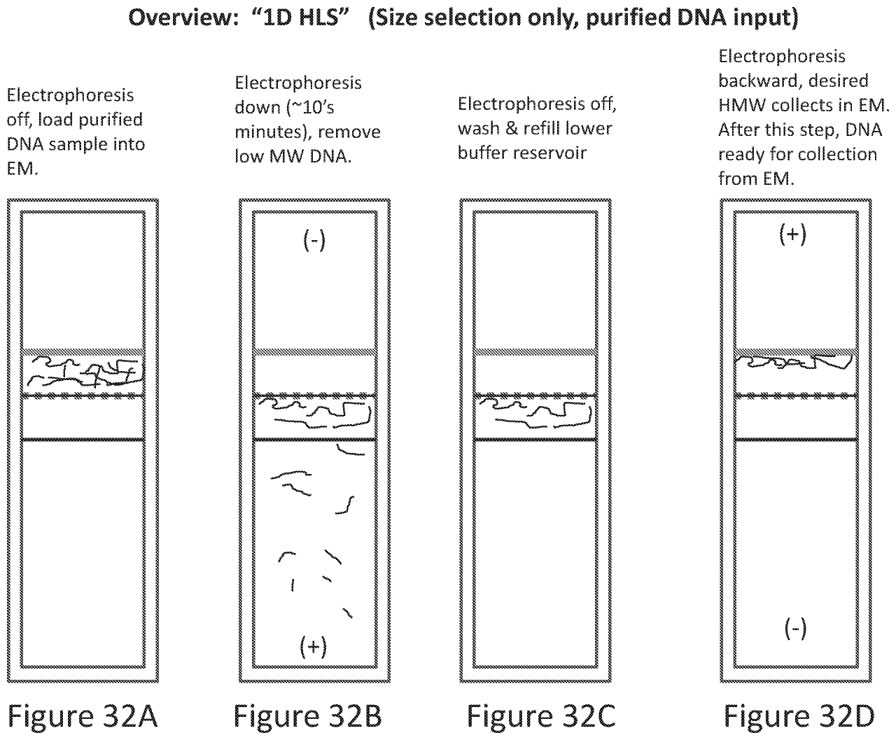

Gel electrophoresis—FIG. 17 (image without lettering shown in FIG. 29), FIG. 18.

This shows that when the desired high molecular weight E coli genomic DNA (center of mass aproximately 30 Kbp) was mixed with undesired low molecular weight DNA (2 log ladder) and then size fractinated, that fragments smaller then about 12 kbp were removed

This demonstrates that the size fractionation cutoff depends on the electrophoretic conditions used.

### Example 4: Isolation of Bacterial DNA

### Device Preparation

Two cassettes were prepared as described in Example 1, except the agarose gel column next to the Durapor membrane is 0.5 cm long.

Bacterial Growth and Conditions for spheroplast formation

Strain MG1655 (ATCC 700926), is propagated on M9 minimal plates with 1% glucose, 1 millimolar Thiamine, 0.2 millimolar magnesium sulfate, 0.1 millimolar calcium chloride, 0.1% 5-fluoroorotic acid, and 20 μg/mL uracil.

Overnight cultures are made by inoculating a single colony into 5-40 mLs of Trypticase soy broth, and allowing the cells to grow overnight at 37 degrees Centigrade with shaking.

Spheroplasts are prepared by incubating E coli cells with lysozyme

Lysozyme (Epicentre, Ready-Lyse™ Lysozyme Solution, catalog number R1804M, 37,500 units/uL) was diluted 1:40 by mixing 2.5 microliters of lysozyme with 100 microliters of TES20+BSA buffer. TES20 is 10 millimolar Tris 7.5; 1 millimolar EDTA; 100 millimolar NaCl; 20% w/v sucrose. TES20+BSA is 1 mL of TES20 plus 5 microliters of BSA (New England Biolabs, Ipswich Mass., 20 milligram/mL)

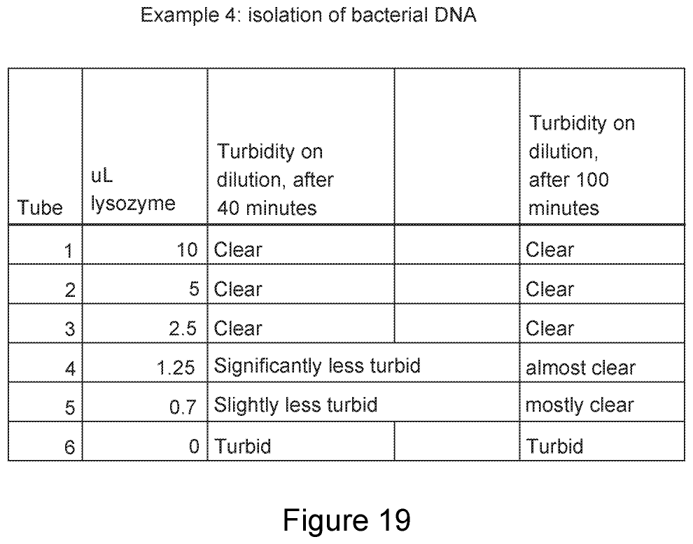

The amount of lysozyme needed for lysis was determined as follows: A series of tubes were prepared as follows: To a 1.7 mL microfuge tube, 800 microliters of ACPS20 buffer (10 millimolar Tris HCl pH 7.5; 5 millimolar EDTA; 20% wt/v sucrose) was added, followed by 500 microliters of the overnight E coli culture. The tube was mixed (vortex mixer) and the cells pelleted by centrifugation (14,000×g one minute). The supernatant was decanted, and the cell pellet re-suspended in 100 microliters ACPS20 by vortexing. As shown in FIG. 19, different amounts of lysozyme were added, and lysis was checked by taking an aliquot and diluting 1:10 into water; unlysed cells formed a turbid solution on dilution, while lysed cells form a clear solution.

Lysis of cells in mixture of lysozyme and Achromopeptidase (per U.S. Pat. No. 4,900,677, which is incorporated herein by reference in its entirety). 100 microliters of a 1 milligram per mL solution of BSA (New England Biolabs, Ipswich Mass.) in water was added to one vial of Achromopeptidase (Sigma catalog # A3422, 25,000 units, 1 milligram)

Aliquots were made and stored at −20 degrees Centigrade.

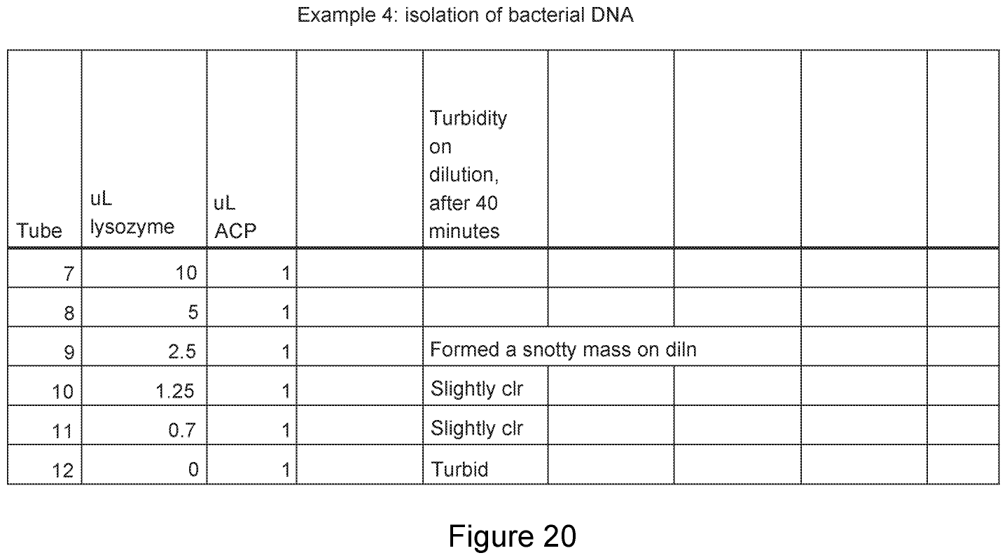

A series of tubes was prepared as above, and as shown in FIG. 20, lysozme and achromopeptidase were added. Lysis is checked as above by dilution into water.

The results show that by itself (tube 12) Achromopeptidase does not lyse cells, but that ACP is synergistic with lysozyme, e.g. tubes 7-9 in FIG. 20 are more highly lysed then tubes 1-3 in FIG. 19.

### Preparation of Spheroplasts and Isolation of DNA

Two tubes of cells were prepared as described above. To tube one, 1.5 microliter of diluted lysozyme was added; to tube 2 was added 1.5 microliter of lysozyme and 1 microliter of ACP. The tubes were vortexed and allowed to sit at room temperature for forty minutes.

### Sample

Elution modules were loaded with a mixture of 100 microliters of spheroplasts and 300 microliters of ACPS20 buffer. After filling the elution modules, 10 microliters was withdrawn and diluted into 190 microliters of QLB (Qubit lysis buffer, 0.5×KBB, 1% weight to volume SDS, 5 millimolar EDTA, 50 millimolar NaCl); the tubes were allowed to sit at room temperature.

As a tracking dye, 1 microliter of 10 milligram/ml phenol red was added to each elution module, and the contents gently mixed with a pipette.

The Spheroplasts were entangled into the agarose coated membrane with electrophoresis (40 V DC, twenty minutes, with the positive electrode on the Durapor side). During this period, the phenol red migrated out of the elution module and into the agarose as a broad band.

To the elution module, 100 microliter of 10% SDS was added, the solution was mixed and electrophoresis continued as above for forty minutes.

After a few minutes, it was observed that the volume of liquid in the elution module was increasing; the porthole in the top of the elution module was sealed with a rubber stopper.

It is believed that the increase in volume after addition of SDS is due to electroendosmosis, and the net change in liquid reflects the net balance of electroendosmosis. Electroendosmosis is due to fixed charges. During the first step, the majority of charges are negative charges on the PES membrane. As a result, the net flow of water is through the PES membrane and out of the elution module.

After addition of SDS, positive proteins, which coat DNA in vivo, are removed. Since the long DNA molecules are immobile, and have a high net negative charge, they serve as an electroendosmotic pump, moving liquid into the chamber.

At the end of the electrophoresis step, the buffer chambers were washed by removing and refilling with 0.5×KBB. The rubber stopper was removed from the elution module and the contents aspirated and saved as Fraction 1.

The elution module was rinsed twice with 0.5×KBB (Fraction 2), and then with 500 microliters of enzyme reaction buffer (Fraction 3). Enzyme reaction buffer (ERB) is 0.5×KBB; 32 milligram/mL hydroxy propyl beta cyclodextrin [ACROS Organics, 97%, catalog #297560250, CAS 128446-35-5]; 10 millimolar Mg(Cl)2; 50 micrograms/mL BSA).

The elution module was then filled with 500 microliters of ERB to which had been added 5 microliters of 20 milligram/mL BSA (New England Biolabs, Ipswich M)); 1.5 microliters of fragmentase enzyme (New England Biolabs, Ipswich Mass.); and 1 microliter of T7 Endonuclease I (New England Biolabs, Ipswich Mass.). After thirty minutes at room temperature, 15 microliters of 500 millimolar EDTA was added to the elution module, the contents were mixed, and the solution removed (Fraction 4). The elution module was rinsed with 0.5×KBB (Fraction 5), and refilled with the same buffer. The digested DNA was recovered by electrophoresis (50 V DC, two minutes, PES side positive; thirty seconds with a pulse train of 4 msec forward/4 msec reverse). The contents of the elution module were removed and saved as Fraction 6/Elution 1

The elution process was repeated two more times, generate Fraction 7/Elution 2 and Fraction 8/Elution 3.

### Qubit Analysis of Fractions

The concentration of DNA in each fraction was measured using a Qubit HS assay.

From the Qubit HS assay of fraction 0, input, the total amount of DNA in the spheroplasts added to the elution module was 40 micrograms.

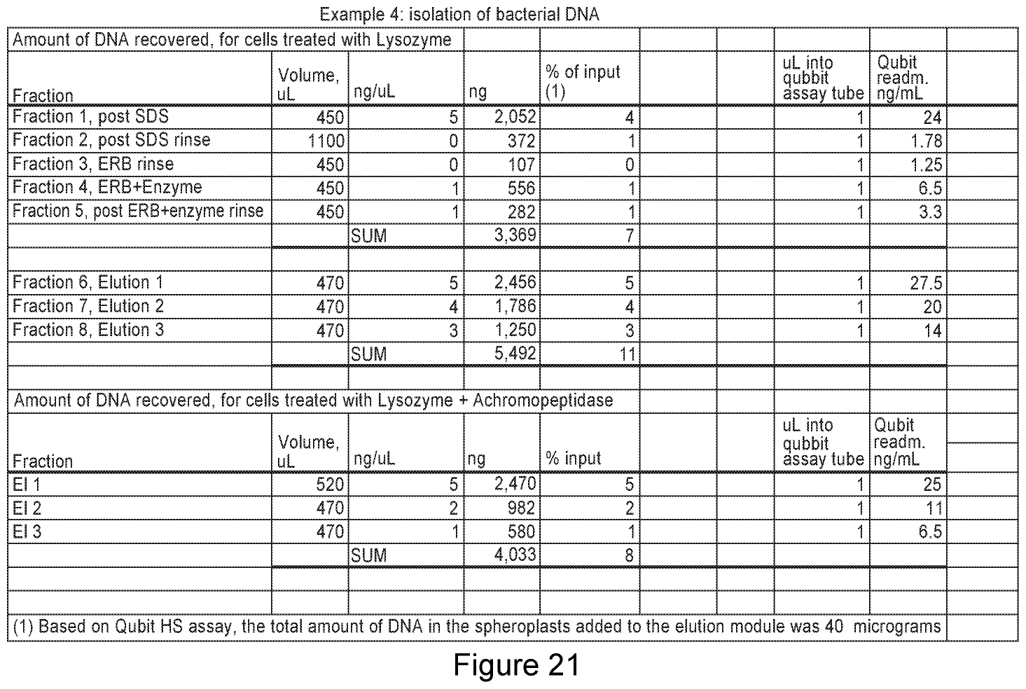

As shown in FIG. 21, the amount of DNA recovered in the elution fractions from cells treated with lysozyme was 5,492 nanograms, 11% of the input.

The amount of DNA recovered in the other fractions was 7%. Very little DNA (1% was recovered in fractions 4 and 5; this shows that after digestion with fragmentase the DNA is still entangled in the agarose and not free to diffuse into the sample compartment.

Similar results were obtained with cells treated with both lysozyme and achromopeptidase (FIG. 21).

### Analysis of DNA by Agarose Gel Electrophoresis

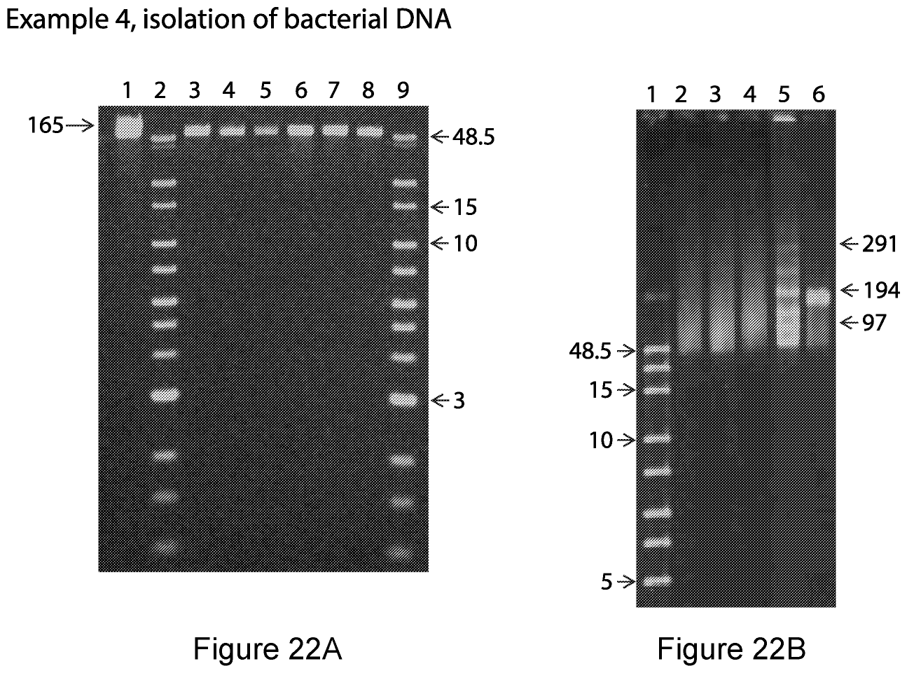

FIG. 22A: analysis of E coli DNA by agarose gel electrophoresis.

0.75% seakem gold agarose (Lonza); 0.5×KBB (Sage); a Pippin Pulse (Sage) was used with the following waveform parameters: 150; 50; 30; 10; 3; 1; 48.

The gel was run for 8 hrs at 80 Volts.

| Lane | Sample |
|---|---|
| 1 | Phage T4 DNA, 166 Kbp (T4 GT7 DNA catalog #318-03971) |
| 2 | New England Biolabs, Ipswich MA) 1 kb extend marker |
| | (catalog # N3239S) |
| 3 | Elution 1 from cells treated with both lysoszyme and |
| | achromopeptidase |
| 4 | Elution 2 from cells treated with both lysoszyme and |
| | achromopeptidase |
| 5 | Elution 3 from cells treated with both lysoszyme and |
| | achromopeptidase |
| 6 | Elution 1 from cells treated with both lysoszyme |
| 7 | Elution 2 from cells treated with both lysoszyme |
| 8 | Elution 3 from cells treated with both lysoszyme |
| 9 | New England Biolabs, Ipswich MA) 1 kb extend marker |
| | (catalog # N3239S) |

The majority of the DNA migrates as a band at the limit mobility, approximately 45 Kbp

FIG. 22B: analysis of E coli DNA by agarose gel electrophoresis.

0.75% seakem gold agarose (Lonza); 0.5×KBB (Sage); a Pippin Pulse (Sage) was used with the following waveform parameters: 300; 100; 30; 10; 30; 10; 45.

The gel was run for 12 hrs at 80 Volts.

| Lane | Sample |
|---|---|
| 1 | New England Biolabs, Ipswich MA) 1 kb extend marker |
| | (catalog # N3239S) |
| 2 | Elution 1 from cells treated with both lysoszyme |
| 3 | Elution 2 from cells treated with both lysoszyme |
| 4 | Elution 1 from cells treated with both lysoszyme + |
| | achromopeptidase |
| 5 | Lambda DNA ladder (Lambda DNA, 48.5 Kb, ligated |
| | following the protcol described in Nucleic Acids |
| | Res. 1990 May 25; 18(10): 3090. |
| 6 | T4 DNA |

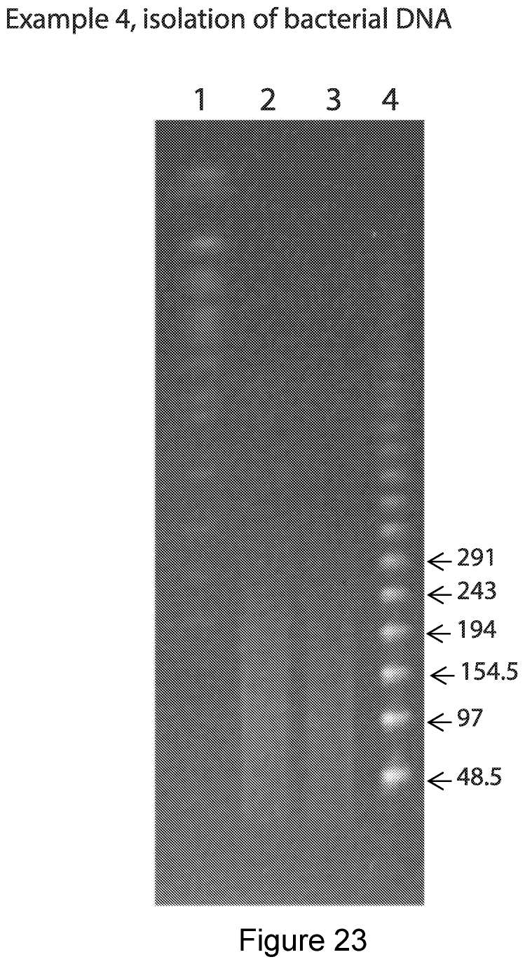

See FIG. 23.

E coli DNA from one D analyzed by agarose gel electrophoresis.

1% SGK gel, 0.5×KBB with BioRad CHEF mapper, program Molecular weight: low 50 K, high 1000 K; Gradient: 6 V/cm; Angle: 120; Run time: 14: 54; Initial switch time: 6.75 s; Final switch time: 1 m 33.69 s; Ramping factor: linear

| Lane | Sample |
|---|---|
| 1 | Yeast Chromosome ladder (New England Biolabs, Ipswich MA)) |
| 2 | Elution 1 from cells treated with lysoszyme |
| 3 | Elution 2 from cells treated with lysoszyme |
| 4 | Lambda Ladder (New England Biolabs, Ipswich MA)) |

### Example 5: Isolation of High Molecular Weight DNA from White Blood Cells

### Device

As shown in FIG. 7A-B, a lid 1 is fitted to a base 2; silicone grease is applied to shelf 4. The lid serves to divide the buffer chambers into separate anonic and cathodic compartments that can communicate only through the elution module.

The lid also serves to define the top surface of the agarose gel.

### Assembly

A solution of Glycerol/EtOH is applied to the PES membrane of an elution module. After the EtOH evaporates, the elution module is filled with 0.5×KBB buffer and examined for leaks. The elution module is then dried by removing the buffer and carefully blotting dry with a paper towel; the elution module is placed in the base.

A small amount of silicone grease is applied to the shelf in the base, and the lid is the added; the assembly is held together with spring clamps. A casting dam is used to form an agarose block next to the Durapor membrane; after the agarose gels, it is trimmed to a 5 mm long block.

The device is then filled with buffer and run for a few minutes at 50 V DC, with the positive electrode on the Durapor side. The current is observed to be 4.3 mA

### White Blood Cells

All steps at 4 degrees centigrade.

White blood cells (also referred to herein as “WBCs”) are prepared from whole blood from goats (Lampire, 3599 Farm School Rd, Ottsville, Pa. 18942) with ACD anticoagulant. To 37 mL cold RBC lysis buffer (1× buffer is 155 millimolar Ammonium Chloride; 10 millimolar NaHCO₃; 1 millimolar Na2EDTA) was added 10 mL of whole blood; tubes were mixed by inversion, and incubated for five minutes at 4 degrees centigrade with occasional mixing. The WBCs are recovered by centrifugation (2,400×g for four minutes.)

The supernatant was decanted and the reddish pellet of white cells washed by re-suspending (vortex) in 20 mL RBC lysis buffer and centrifugation at 2,200×g for two minutes.

The wash step is repeated 2-3× until the cell pellet has only a trace of red color.

The cells are re-suspended in 1.5 mL of FSE (50% v/v Sage Ficoll loading buffer; 80 milligram/mL sucrose; 10 millimolar EDTA) and filtered (40 micron sterile cell strainer, Fisher Scientific catalog #22363547).

The white blood cells can be stored at 4 degrees centigrade for several days. If the cell suspension is not a homogeneous, creamy solution it is vortexed or re-filtered. If re-filtered, the concentration of cells or DNA needs to be re-measured.

Quantification of DNA with a Qubit HS Assay.

Gently mix the WBCs by swirling the tube, and transfer 10 microliters to a microfuge tube; then add 190 microliters of Qubit lysis buffer and mix by pipetting; the solution will become snotty. Incubate at 58 degrees centigrade for ten minutes. Cool to room temperature, add 600 microliter of TE and vortex full speed for ten seconds.

Assay 0.5 to 1 microliter of the lysed cell mix with a Qubit HS assay. The expected concentration of DNA is 200-300 nanogram/uL

### Quantification of Cells by Cell Counting

A BioRad TC20 automated cell counter was used to determine total cell counts and percent viability with trypan blue, following the vendor's directions.

### Load Cells and Isolate DNA

To prepare the sample, mix 75 microliters WBCs (69,000 cells/uL, nominal 416 nanogram per microliter of DNA, assuming 6 picogram/cell) with 350 microliters of SEK buffer (0.5×KBB; 5 millimolar EDTA; 80 milligram/mL sucrose; 10 microgram/mL phenol red) and add to the elution module.

To determine the concentration of DNA in this solution (the input), two 10 microliter aliquots were withdrawn and added to 190 microliters of qubit lysis buffer; the solutions were vortexed and stored at room temperature until assayed with a Qubit HS assay.

The sample was Electrophoresed for thirteen minutes at 50 V DC, with the positive electrode on the Durapor side. After thirteen minutes, 150 microliter of 10% SDS was added to the elution module and the solution mixed gently. A 20 microliter aliquot (Fraction 0) was taken and added to 190 microliter of qubit lysis buffer to determine the concentration of DNA.

The elution module was sealed with a stopper, and electrophoresis was continued for another ten minutes at 50 V DC. The buffer in the buffer chambers was replaced, and electrophoresis continued for another ten minutes.

The contents of the elution module were removed, and 12 microliter of 500 millimolar EDTA was added; this is Fraction 1, post SDS. The elution module was rinsed with KBB and the liquid saved as Fraction 2, post SDS rinse.

The buffer chambers were rinsed three times to remove SDS, and fresh buffer was placed in the buffer chambers. The elution module was rinsed with 500 microliter of ERB (Fraction 3, ERB rinse). DNA was digested by adding 500 microliter of ERB, to which had been added 5 microliters of 20 mg/mL BSA (New England Biolabs, Ipswich M)), 1.5 microliters of Fragmentase enzyme (New England Biolabs, Ipswich Mass.), and 0.5 microliter of T7 Endonuclease I (New England Biolabs, Ipswich Mass.); incubation was for ten minutes at 37 degrees Centigrade (the entire device was placed on a thermostatted aluminum plate (Benchmark “myBlock” dry block heater unit). At the end of the incubation, 15 microliter of 0.5 M EDTA was added to the elution module, the contents were gently mixed, and the solution was aspirated from the ELUTION MODULE and saved as Fraction 4, ERB.

DNA was then recovered by electroelution; the elution module was filled with 500 microliters of 0.5×KBB and 5 microliters of 0.5 M EDTA, and voltage applied (50 V DC, 90 seconds, with the positive electrode on the Durapor side); the solution was recovered as Fraction 5, elution 1.

The elution module was refilled with 0.5×KBB, and DNA eluted for two minutes, 50 V DC.

The solution was saved (Fraction 6, elution 2). The elution module was refilled, and electrophoresis applied for four minutes, followed by five seconds of 25 V DC with the positive electrode on the Durapor side (reverse current pulse, to back DNA off of the PES membrane). The device was allowed to sit at room temperature, covered to avoid evaporation, overnight; the material in the ELUTION MODULE was recovered the next day (Fraction 7, elution 3).

### Results

The amount of DNA in each fraction was determined using a Qubit HS assay.

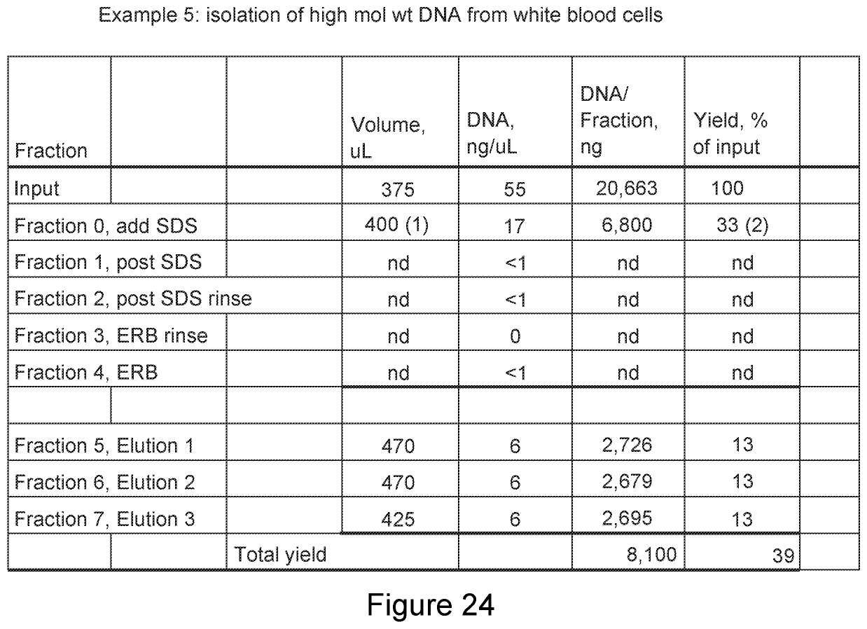

See FIG. 24.

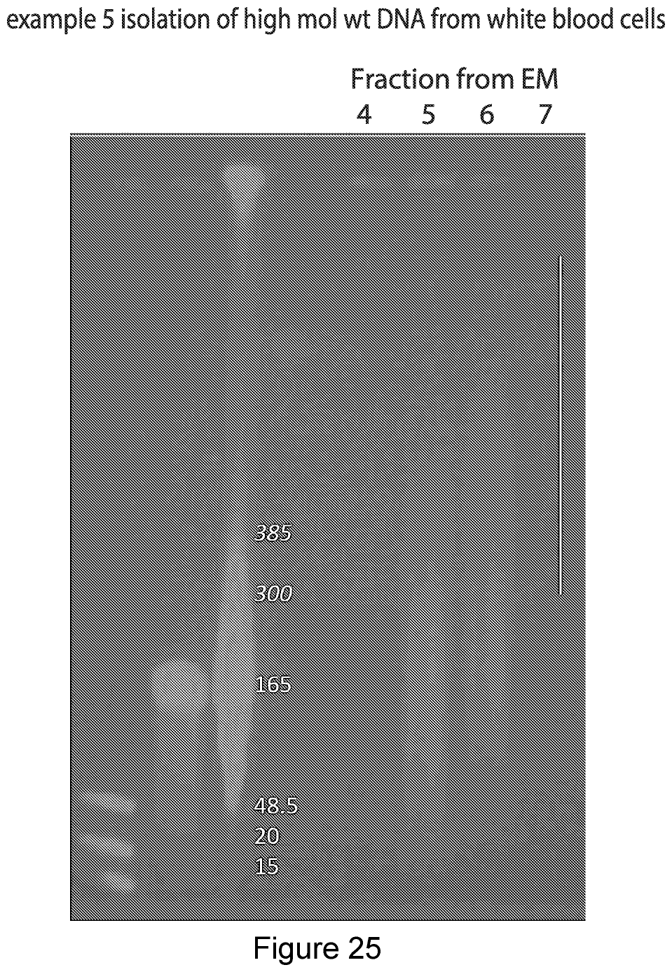

As shown in FIG. 25, 20,663 ng of DNA were loaded into the elution module, and that after thirteen minutes of electrophoresis at 50 V DC, Durapor side positive, only 33% of the DNA (6,800 ng) was present in the elution module in a form that could be recovered after adding SDS. This suggests that the DNA was bound or entangled in some form on either the Durapor or agarose, or both, and that this DNA was not released by washing (Fractions 2, 3) or treatment with Enzyme (Fraction 4).

The results show that 39% (8,100 ng) of the input DNA could be recovered in the elution.

The size of the DNA in each fraction was determined by agarose gel electrophoresis.

30 microliters of fractions 4, 5, 6, and 7 were analyzed on a Pulse field agarose gel (BioRad CHEF mapper; program is default for separation of 50 to 1,000 KBp fragments, with a time factor of 0.5; the gel is 0.75% seakem gold agarose (Lonza) in 0.5×KBB buffer (Sage); the running temperature is 14 degrees Centigrade.

| Lane | sample |
|---|---|
| 1 | 1 Kb extend ladder (New England Biolabs, Ipswich MA)) |
| 2 | T4 phage DNA |
| 3 | Ladder of Lambda phage DNA (New England Biolabs, Ipswich |
| | MA)) |
| 4 | Empty |
| 5 | Fraction 4, enzyme |
| 6 | Fraction 5, first elution (90 seconds) |
| 7 | Fraction 6, 2^{nd} eltution (2 min) |
| 8 | Fraction 6 3^{rd} elution (4 min) |

The results show that high molecular weight DNA, with a center of mass at approximately 160 (Fraction 5) to 300 kb (fraction 6) was obtained. In fraction 7, some DNA is visible at limit mobility (˜2 Mbp in this gel system).

### Example 6: Isolation of High Molecular Weight DNA from White Blood Cells

In this example, rapid, high yield recovery of DNA from white blood cells, using a device configuration where there is a single buffer chamber, is demonstrated—that is, there is no barrier between the anodic and cathodic buffer chambers.

### Device Assembly

As shown in FIG. 26A, an elution module was inserted in the base, and a casting dam was placed manually approximately 5 mm from the elution module; agarose was added with a pipet to fill the space up to the lid shelf

After the agarose gels, the dam was removed and buffer (0.5×KBB) was added to fill the buffer chambers almost up to the top of the elution; the buffer flows freely around the side of the ELUTION MODULE on the shelf.

The ELUTION MODULE is filled with buffer prior to use.

To ensure that the elution module is firmly in place, a spring clamp was added, as shown in FIG. 26B.

Device in use; the base rests on metal blocks for cooling; two electrode holders with platinum wire are shown; a clamp holds the elution module in place

Buffer can flow between the two electrodes around the side of the elution module.

### Sample

WBCs from goat whole blood were prepared as described in Example 3. Two aliquots of Cells were diluted into TBS and counted with a BioRad cell counter, following manufacturer's instructions. The results were:

| | | |
|---|---|---|
| SampleCells/uL | % viable | DNA, ng microliter(1) |

| | | | |
|---|---|---|---|
| 1 | 79,000 | 47 | |
| 2 | 108,000 | 47 | |
| Avg | 93,500 | 47 | 561 |

(1)Assuming 6 pg of DNA per cell

Cells were stored overnight at 4 degrees Centigrade, and then recounted

| | | |
|---|---|---|
| SampleCells/uL | % viable | ng/uL |

| | | | |
|---|---|---|---|
| 1 | 84,000 | 35 | |
| 2 | 80,000 | 35 | |
| Avg | 82,000 | 35 | 490 |

41 microliters of cells (20 microgram of DNA) were mixed with 290 microliters of SEK Buffer (0.5×KBB; 5 millimolar EDTA; 80 milligram/mL sucrose; 10 microgram/mL phenol red) and added to the elution module. To determine the starting concentration of DNA (“input”), two 10 microliter aliquots were taken and each aliquot was diluted into 190 microliters of QLB (Qubit lysis buffer, 0.5×KBB, 1% SDS, 5 millimolar EDTA, 50 millimolar NaCl); samples were mixed and stored at room temperature.

Cells were electrophoresed into the agarose coated Durapor; electrophoresis was 50 V DC, with the positive electrode on the Durapor side of the ELUTION MODULE. The current was 6.2 mA.

After eight minutes thirty seconds, electrophoresis was paused; the phenol red dye in the SEK had migrated out of the ELUTION MODULE and halfway thru the agarose as a broad band.

To the elution module was added 80 microliter of 10% SDS and 160 microliter of 0.5×KBB; the elution module contents were mixed gently and the porthole sealed with a rubber stopper.

Restart electrophoresis at 50 V; the current was 9.8 mA; switch to 40 V; the current was 7.6 mA.

It is observed that addition of SDS to the elution module usually causes the current to rise. Since the joule heat is proportional to the current, the voltage is decreased to avoid overheating.

Continue electrophoresis for a total time of twenty minutes, then pause.

Remove the rubber stopper, and take 10 microliter from the ELUTION MODULE; add to 90 microliter QLB as Fraction 1, “post SDS lysis step”.

Rinse the buffer chambers 3× with fresh buffer to remove SDS from the device.

Rinse the ELUTION MODULE 2× with buffer, and save as Fraction 2, “post SDS rinse”.

Rinse a 3^{rd} time with KBB, and save as Fraction 3, “post SDS rinse”.

Mix 500 microliter of ERB (5×KBB; 32 milligram/mL hydroxy propyl beta cyclodextrin; 10 millimolar Mg(Cl)2; 50 microgram/mL BSA) with 5 microliter of 20 mg/mL BSA (New England Biolabs, Ipswich Mass.)); 1 microliter of T7 endonuclease I (New England Biolabs, Ipswich Mass.)); and 1 microliter of Fragmentase (New England Biolabs, Ipswich Mass.)); add the ERB/enzyme cocktail to the ELUTION MODULE and let sit room temperature for 36 minutes.

Recover the solution; add 15 microliter of 0.5 M EDTA, and save as Fraction 4 “Enzyme mix”.

To 500 microliter of 0.5×KBB add EDTA to 25 millimolar and DTT to 5 millimolar; add to the ELUTION MODULE and let sit for five minutes; recover and save as Fraction 5, “Post enzyme wash”.

Add 400 microliter of KBB and elute DNA with electrophoresis (two minutes, 40 V DC, Durapor side negative). Recover the sample and save as Fraction 6, “elution 1”

Repeat the elution process; observe that the second elution is “snotty,” and indication of high molecular weight DNA; save as Fraction 7, “elution 2”

400 microliter of KBB was added to the elution module, and DNA eluted with the following program, using a Pippin pulse controller.

| Step | Electrophoresis |
|---|---|
| 1 | 50 V DC 2 min |
| 2 | 50 V with a pulse of 4 msec fwd/4 msec rev (forward is with |
| | the PES side Positive) |
| 3 | 50 V DC 2 min |
| 4 | 50 V pulse 2 min |
| 5 | 50 V DC 2 min |
| 6 | 50 V pulse, 12 minutes |

Recover the material in the elution module as Fraction 8, “elution 3”

### Results

The amount of DNA in each fraction was determined using a Qubit HS assay.

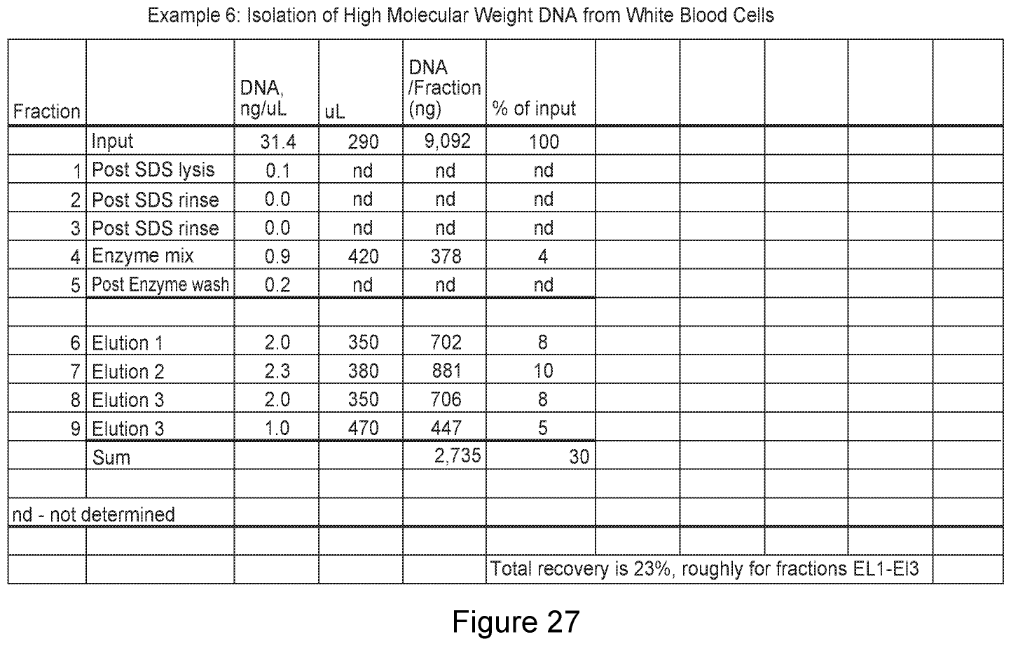

See FIG. 27.

### CONCLUSION

The data shows that we can recover 30% of the input DNA, using a format where there is a single buffer chamber that is not divided into an anodic and cathodic compartment.

Further, the data shows that the volume of liquid in the elution module sample compartment changes during electrophoresis. During the first step, when cell are migrating to, and becoming entangled in the agarose coated membrane, the volume in the sample compartment decreases.

During the second step, after SDS is added, it is observed that after a few minutes the volume starts to increase.

We believe that changes in volume are due to electro endosmosis. In the first step, we believe that the majority of fixed charges are negative charges on the PES membrane; this causes water to be pumped through the PES and out of the elution module.

After addition of SDS, chromosomal DNA is freed from positively charged proteins (e.g., histones); as a result, the immobile, entangled chromosomal DNA acts as a fixed negative charge, pumping buffer into the elution module.

### Example 7, Prophetic: A Device Designed to Run Multiple Samples Simultaneously

One advantage of our system for purifying DNA is that it is adaptable to handle large numbers of samples.

Large numbers of samples are common (Ledford, Heidi, “AstraZeneca launches project to sequence 2 million genomes,” Nature: International Weekly Journal of Science, 532, 427, Apr. 28, 2016, incorporated herein by reference in its entirety) thus there is a need for systems that can handle hundreds or thousands of samples reliably, rapidly, and at low cost.

The demand for large sample capacity is evident from the large number of products sold for laboratory automation (e.g., Tecan Liquid Handling and Robotics product lines, incorporated herein by reference in its entirety).

Such products automate steps such as liquid handling, moving disposables such as cassettes, and collection and analysis of data.

FIGS. 12-13 show a cassette configured to hold four elution modules, which allows for analysis of four samples at a time. The cassette is fitted with four elution modules, and four agarose gels are cast, one for each elution module; buffer is added and the cassette is sealed. Procedures for automated casting of agarose gels, filling of cassettes with buffer and cassette sealing are utilized by Sage Science for production of cassettes for the Pippin and ELF instruments (e.g., SageHLS High Molecular Weight Library System, PippinHT DNA Size Selection System, SageELF Sample Fractionation System, BluePippin Size Selection System, Pippin Prep DNA Size Selection System, SageHLS, PippinHT, SageELF, Pippin Prep, BluePippin, all of which are incorporated herein by reference in its entirety).

Cassettes, filled with buffer and sealed, are suitable for storage until needed.

Automation of liquid handling steps for devices such as the cassette shown in FIG. 13 can be accomplished with liquid handling robots (e.g., Tecan Freedom EVO® Series, incorporated herein by reference in its entirety). Such robots can be configured to hold various samples and reagents, and to deliver reagents to a disposable cassette, such as the one shown in FIG. 13.

This allows for the automation of all the liquid handling steps required for isolation of DNA.

In addition, a means of providing electrodes may be used. Sage Science has demonstrated (HLS) instruments which have electrodes on a movable lid

### Example 8, Electrophoresis of SDS in a Cassette with Three Buffer Chambers

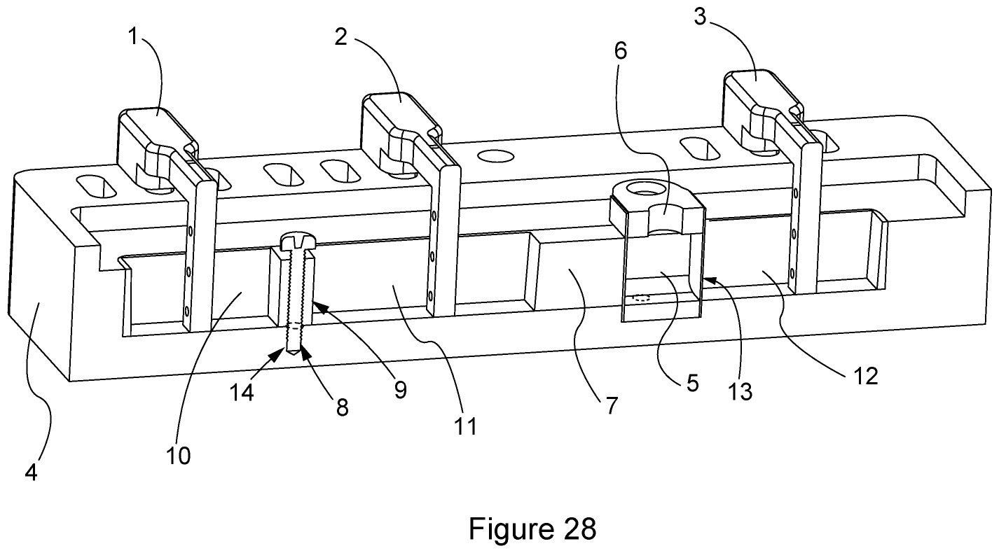

See FIG. 28.

A base 4 was fitted with an elution module 13 with a sample compartment 5 and a porthole 6. A block of agarose 7 was cast next to the elution module using a casting dam as described in Example 6.

An M2 screw, nylon, 8, was added to the threaded hole 14, and then two casting dams were placed in the base 4 around the screw 8, and molten agarose was added; after the agarose hardened to form a block 9, the casting dams were removed, and the buffer chamber 11 was filled with buffer.

After twenty minutes, it was observed that very little liquid was present in buffer chamber 10. We infer that the agarose block 9 forms a seal separating buffer chambers 10 and 11.

A similar experiment, but without the screw 8 and screw hole 14 resulted in a block of agarose 8 that did not form a seal.

The sample compartment 5 and the buffer chambers 10, 11, 12 were filled with buffer, and electrodes 1, 2, 3 were added.

A small amount of tracking dye (Xylene Cyanol) was added to the sample compartment so that the solution was easily visible to the naked eye.

A Pippin pulse power supply was used to supply 50 V DC between electrodes 2 and 3, with electrode 2 positive. It was observed that the Xylene Cyanol tracking dye moved from the sample compartment 2, through the agarose block 7 and into the buffer chamber 11.

A pippin pulse power supply was then connected to electrodes 1 and 2, with electrode 1 positive, and 50 V DC was applied. It was observed that the tracking dye moved out of buffer chamber 11, through agarose block 7, and into buffer chamber 10; the solution in buffer chamber 11 changed from dark blue to clear.

We infer that this sequential electrophoresis allowed us to transport, by electrophoresis, negatively charged Xylene Cyanol molecules (xylene cyanol has a net negative charge, Ter Ming Tan, Timothy, et al., “Gel Electrophoresis: DNA Science without the DNA!,” Biochemistry and Molecular Biology Education, vol. 35, No. 5, pp. 342-349, 2007, incorporated herein by reference in its entirety) from the sample compartment and sequester them in the buffer chamber 10.

### Example 9: Prophetic, Isolation of DNA without Washing of the Cassette

In Example 6, we describe isolation of high molecular weight DNA. In that example, SDS is used to deproteinize the cells, and the SDS, and SDS coated protein, is removed from the cassette so that enzymatic digestion can occur, and so that the SDS and SDS coated protein do not contaminate the purified DNA during the elution step.

During this process, washing was used to remove SDS and SDS coated protein from the buffer chambers, so that the SDS and SDS coated protein would not contaminate the DNA during elution.

In this example we use electrophoretic transport to sequester the SDS and SDS coated protein, so that washing is not needed.

A cassette is prepared as described with respect to FIG. 28, and a sample is prepared and loaded as described in Example 6.

Electrophoresis to entangle cells in the agarose coated membrane is as described in Example 6, except that electrodes 2, 3 (FIG. 28) are used. SDS is added as described in Example 6, and electrophoresis is used to cause cell lysis and deproteinization, as described in Example 6, except that electrodes 2, 3 are used.

Current (50 V DC) is then applied between electrodes 1, 2, with electrode 1 positive, for 1 hour to transport SDS and SDC coated protein, from buffer chamber 11, through agarose gel 9 and into buffer chamber 10.

The DNA, which is entangled in the agarose coated membrane, is then treated with fragmentase, as described in Example 6, except that the buffer chambers are not washed to remove SDS. The DNA is recovered as describe in Example 6, except that electrodes 2, 3 are used, with electrode 3 positive.

This example shows purification of high molecular weight DNA from a cellular sample without the need to wash the cassette to remove SDS or other contaminants.

### Other Applications

This application is also related to:
U.S. application Ser. No. 15/183,097, filed Jun. 15, 2016
U.S. application Ser. No. 14/297,001, filed Jun. 5, 2014
U.S. application Ser. No. 13/751,606, filed Jan. 28, 2013
U.S. application Ser. No. 12/760,548, filed Apr. 14, 2010
U.S. application Ser. No. 12/576,148, filed Oct. 8, 2009
U.S. Provisional Application No. 61/150,243, filed Feb. 5, 2009
U.S. Provisional Application No. 61/195,566, filed Oct. 8, 2008
U.S. application Ser. No. 15/464,278, filed Mar. 20, 2017
U.S. application Ser. No. 14/051,300, filed Oct. 10, 2013
U.S. Provisional Application No. 61/766,910, filed Feb. 20, 2013
U.S. Provisional Application No. 61/713,916, filed Oct. 15, 2012
U.S. Provisional Application No. 61/713,156, filed Oct. 12, 2012
U.S. application Ser. No. 15/519,516, filed Apr. 14, 2017
PCT Application No. PCT/US2015/055833, filed Oct. 15, 2015,
U.S. Provisional Application No. 62/183,514, filed Jun. 23, 2015
U.S. Provisional Application No. 62/064,454, filed Oct. 15, 2014

The aforementioned applications are all expressly incorporated by reference herein in their entireties.

Any and all references to publications or other documents, including but not limited to, patents, patent applications, articles, webpages, books, etc., presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices, including any and all elements corresponding to molecular processing. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference/prior art by specifically lacking one or more elements/features of a system, device and/or method disclosed in such prior art. In other words, claims to certain embodiments may contain negative limitation to specifically exclude one or more elements/features resulting in embodiments which are patentably distinct from the prior art which include such features/elements.

We claim:

1. A disposable cassette for an automated molecular processing apparatus, comprising:
a base housing;
a central channel arranged in the housing; and
an elution module configured to be received in central channel and to divide the central channel into a first chamber and a second chamber;
wherein the elution module comprises:
an elution module housing having a proximal side, a distal side and an elution module channel passing from the proximal side to the distal side;
a first membrane attached to a proximal side of the elution module, the proximal side of the elution module traversing the central channel and forming an end of the first chamber,
a second membrane attached to a distal side of the elution module, the distal side of the elution module parallel to the proximal side of the channel and forming an end of the second chamber; and
a porthole in fluid communication with the elution module channel and configured for receiving a sample.

2. The cassette of claim 1, further comprising at least two electrode holders configured to fit within slots in the base and configured to receive electrodes such that at least one electrode is arranged within the first chamber and at least one electrode is arranged within the second chamber.

3. The cassette of claim 1, wherein the first membrane is more porous than the second membrane.

4. The cassette of claim 1, wherein the second membrane is configured to retain nucleic acid molecules.

5. The cassette of claim 4, wherein the nucleic acid molecules comprise DNA

6. The cassette of claim 1, wherein the elution module is comprised of plastic, and wherein the first membrane and the second membrane are heat bonded to the plastic of the proximal and distal sides of the elution module, respectively.

7. The cassette of claim 1, wherein the first and second membranes are configured to substantially block fluid flow.

8. The cassette of claim 2, wherein the first and second membranes are configured to pass molecules upon application of current thereto.

9. The cassette of claim 1, wherein the first chamber and the second chamber contain a buffer solution.

10. The cassette of claim 1, wherein the elution module further comprises openings configured to receive fasteners to affix the module to the cassette.

11. The cassette of claim 1, wherein the elution module is configured for clamping attachment to the cassette.

12. An elution module for a disposable cassette used in an automated molecular processing apparatus, wherein the cassette includes a central channel arranged therein for which the elution module is placed to divide the channel into a first chamber and a second chamber, the module comprising:
a housing having a proximal side, a distal side and an elution module channel passing from the proximal side to the distal side;
a first membrane attached to a proximal side of the elution module, the proximal side of the elution module forming an end of the first chamber,
a second membrane attached to a distal side of the elution module, the distal side of the elution module parallel to the proximal side of the channel and forming an end of the second chamber; and
a porthole in fluid communication with the elution module channel and configured for receiving a sample.

13. The module of claim 12, wherein the porosity of the first membrane is greater than that of the second membrane.

14. The module of claim 12, wherein the second membrane is configured to retain nucleic acid molecules.

15. The molecule of claim 14, wherein the nucleic acid molecules comprise DNA.

16. The module of claim 12, wherein housing is comprised of plastic and the first membrane and the second membrane are heat bonded to respective sides of the housing.

17. The module of claim 12, wherein the first and second membranes are configured to substantially block fluid flow.

18. The module of claim 12, wherein the first and second membranes are configured to pass molecules upon the application of current thereto.

19. The module of claim 12, further comprising openings configured to receive fasteners to affix the module to the cassette.

20. The module of claim 12, wherein the housing is configured for clamping attachment to the cassette.

21. A method for preparing a cassette, comprising:
providing:
a base, the base having a central channel having a first end and a second end,
an elusion module, the elusion module having a central plastic piece, a first membrane attached to a first side of the central plastic piece, and a second membrane attached to a second side of the central plastic piece,
at least a first electrode holder and a second electrode holder, the first electrode holder and the second electrode holder each having a wire connected thereto,
a casting dam configured to block a portion between the first end of the central channel and the first membrane, and
a cover configured to cover at least a portion of the central channel;
attaching the elusion module to the base, the elution module spaced apart from the first end and the second end, wherein the first membrane faces the first end of the central channel and the second membrane faces the second end of the central channel;
placing the casting dam to abut the first end of the central channel to create a gap between a distal end of the casting dam and the first membrane;
casting the gap by filling said gap with agarose and allowing the agarose to gel;
removing the casting dam to reveal a portion of the central channel between the first end and the agarose gel;
attaching the first electrode holder between the first end of the central channel and the first membrane and the second electrode holder between the second end of the central channel and the second membrane;
filling the portion of the channel with electrophoresis buffer;
filling an area between the second membrane and the second end of the central channel with electrophoresis buffer; and
attaching the cover to the base.

22. The method of claim 21, further comprising:
inserting a sample into the elution module, the sample comprising targets molecules;
applying a current via the electrode holders, the current causing at least the target molecules to move towards the first membrane; and
collecting the target molecules at or near the first membrane.
